(19) United States
(12) Patent Application Publication
(10) Pub. No.: US 2021/0030837 A1
(43) Pub. Date: Feb. 4, 2021

(54) ANGIOGENESIS INHIBITOR AND SCREENING METHOD FOR ANGIOGENESIS INHIBITORS

(71) Applicant: TOKYO WOMEN'S MEDICAL UNIVERSITY, Tokyo (JP)

(72) Inventors: KATSUHISA MATSUURA, Tokyo (JP); SHINAKO AOKI, Tokyo (JP); SATORU SAKAMOTO, Tokyo (JP); TATSUYA SHIMIZU, Tokyo (JP)

(73) Assignee: TOKYO WOMEN'S MEDICAL UNIVERSITY, Tokyo (JP)

(21) Appl. No.: 16/964,170

(22) PCT Filed: Jan. 24, 2019
(86) PCT No.: PCT/JP2019/002370

§ 371 Date: Jul. 22, 2020

(30) Foreign Application Priority Data
Jan. 25, 2018 (JP) 2018-010489

(51) Int. Cl. A61K 38/15; A61K 35/33; C12Q 1/06; G01N 33/15

(52) U.S. Cl. CPC A61K 38/15; G01N 33/15; C12Q 1/06; A61K 35/33

(57) ABSTRACT

The present invention provides an angiogenesis inhibitor containing as an active ingredient LYPD1 protein or a derivative thereof, a part thereof, or a vector expressing the same, or a cell expressing the same. The present invention also provides a screening method for angiogenesis inhibitors that enhance the expression of LYPD1 protein wherein the method includes (i) a step for treating a first cell by a test substance and culturing and (ii) a step for detecting the expression level of LYPD1 protein from the first cell and comparing with the level of LYPD1 protein of an untreated first cell.

### FIELD

The present invention relates to an angiogenesis inhibitor comprising as an active ingredient LYPD1 protein, a derivative thereof, a part thereof, a vector for expressing the same, or a cell expressing the same. The present invention also relates to a method for screening angiogenesis inhibitors that enhance the expression of LYPD1 protein.

### BACKGROUND

Angiogenesis is the formation of new capillaries from pre-existing capillaries in tissues or organs. Normal angiogenesis occurs in limited situations, such as during development of embryo and fetus, growth of placenta, formation of corpus luteum, maturation of uterus, and healing of wound, and stops when the required situation is achieved. Thus, to avoid excessive angiogenesis, it is tightly regulated, for example, by angiogenesis regulating factors (NPL 1).

Diseases associated with abnormal angiogenesis include inflammatory diseases such as arthritis, ophthalmic diseases such as diabetic retinopathy, dermatologic diseases such as psoriasis, and solid malignant tumors. Primary and metastatic solid malignant tumors are known to induce angiogenesis around them to supply nutrients and oxygen required for their proliferation and growth (NPLs 2 and 3). Furthermore, angiogenesis increases the chance of metastasis of solid malignant tumors.

As an effective therapy for such solid malignant tumors, reduction of angiogenesis has been attempted. For example, reports have been made that attempts of administration of inhibitors against angiogenic growth factors, such as anti-VEGF antibodies, result in prolongation of survival (NPLs 4 to 6). However, inhibition of angiogenesis regulating factors will cause, for example, systemic vascular endothelial dysfunction, resulting in adverse effects such as hypertension and thrombogenesis.

Angiogenesis has not only acceleratory but also inhibitory mechanisms. Cancer therapies by activation of inhibitory mechanisms have not yet become the standard of care, and searches for new therapies continue.

### CITATION LIST

### Non-Patent Literature

[NPL 1] Folkman, J. Angiogenesis in cancer, vascular, rheumatoid and other disease. J. Nature Med. (1995) 1: 27-31
[NPL 2] Folkman, J Tumor angiogenesis: therapeutic implications. New Engl. J. Med., (1971) 285: 1182-1186
[NPL 3] Folkman, J. Angiogenesis. J. Biol. Chem. (1992) 267: 10931-10934
[NPL 4] Tewari K S., et al., Improved survival with bevacizumab in advanced cervical cancer. N Engl J Med. (2014) Feb. 20; 370 (8): 734-43.
[NPL 5] Yang J C., et al., A randomized trial of bevacizumab, an anti-vascular endothelial growth factor antibody, for metastatic renal cancer. New Engl. J. Med. (2003) Jul. 31; 349 (5): 427-34.
[NPL 6] Bear H D., et al., Bevacizumab added to neoadjuvant chemotherapy for breast cancer. New Engl. J. Med. (2012) Jan. 26; 366 (4): 310-20.

### SUMMARY

### Technical Problem

An object of the present invention is to provide a new angiogenesis inhibitor for use in treatment of angiogenesis-related diseases.

### Solution to Problem

To achieve the object, the present inventors studied from various angles. Surprisingly, the inventors have found that LYPD1 protein is a novel agent that reduces angiogenesis, thereby completing the present invention. Accordingly, the present invention provides the following inventions.

[1] An angiogenesis inhibitor comprising as an active ingredient LYPD1 protein or a derivative thereof, or a part thereof; or a vector for expressing the same; or a cell expressing the same.

[2] The angiogenesis inhibitor according to [1] for use in treatment or prevention of an angiogenesis-related disease.

[3] The angiogenesis inhibitor according to [2], wherein the angiogenesis-related disease is solid cancer, diabetic retinopathy, age-related macular degeneration, retinopathy of prematurity, corneal graft rejection, neovascular glaucoma, erythroderma, proliferative retinopathy, psoriasis, hemophilic arthropathy, capillary proliferation in atherosclerotic plaques, keloid, wound granulation, vascular adhesion, rheumatoid arthritis, osteoarthritis, an autoimmune disease, a Crohn's disease, restenosis, atherosclerosis, intestinal adhesion, ulcer, liver cirrhosis, glomerulonephritis, diabetic nephropathy, malignant nephrosclerosis, thrombotic microangiopathy, organ graft rejection, glomerulopathy, diabetes mellitus, inflammation, or a neurodegenerative disease.

[4] The angiogenesis inhibitor according to [3], wherein the solid cancer is cervical cancer, lung cancer, pancreatic cancer, non-small-cell lung cancer, liver cancer, colon cancer, osteosarcoma, skin cancer, head cancer, neck cancer, cutaneous melanoma, intraocular melanoma, uterine cancer, ovarian cancer, rectal cancer, liver cancer, brain tumor, bladder cancer, gastric cancer, perianal gland cancer, colon cancer, breast cancer, fallopian tube cancer, endometrial cancer, vaginal cancer, vulvar cancer, Hodgkin's lymphoma, esophageal cancer, small intestine cancer, endocrine cancer, thyroid cancer, parathyroid cancer, adrenal cancer, soft tissue sarcoma, urethral cancer, penile cancer, prostate cancer, bladder cancer, kidney cancer, ureter cancer, renal cell cancer, renal pelvic cancer, central nervous system (CNS) tumor, primary CNS lymphoma, spinal cord tumor, brainstem glioma, or pituitary adenoma.

[5] The angiogenesis inhibitor according to any one of [1] to [4], wherein the LYPD1 protein has a sequence selected from SEQ ID NOS: 1 to 14 and 19, or has at least 85% sequence identity with a sequence selected from SEQ ID NOS: 1 to 14 and 19.

[6] The angiogenesis inhibitor according to any one of [1] to [5], wherein the cell expresses a higher amount of LYPD protein than skin-derived fibroblasts.

[7] The angiogenesis inhibitor according to [6], wherein the cell is a heart-derived fibroblast.

[8] A method for screening angiogenesis inhibitors that enhance the expression of LYPD1 protein, comprising:

a step (i) of treating and culturing a first cell with a test substance; and

a step (ii) of detecting the expression level of LYPD1 protein in the first cell and comparing it with that of an untreated first cell.

[9] The method according to [8], wherein the first cell is a fibroblast derived from skin, esophagus, testis, lung, or liver.

[10] The method according to [8] or [9], further comprising:

a step (iii) of selecting a test substance that enhance the expression of LYPD1 protein as compared with the level of LYPD1 protein in the untreated first cell in the step (ii);

a step (iv) of adding the test substance to a cell population comprising a second cell and a vascular endothelial cell and/or precursor cell, and culturing the cell population; and

a step (v) of detecting vascular endothelial networks formed by the vascular endothelial cell and/or precursor cell.

[11] The method according to [10], wherein the second cell is a fibroblast derived from skin, esophagus, testis, lung, or liver.

### Advantageous Effects of Invention

According to the present invention, angiogenesis can be inhibited, and then angiogenesis-related diseases can be treated or prevented. In addition, according to the present invention, a novel angiogenesis inhibitor can be obtained that can be used for treatment or prevention of angiogenesis-related diseases.

### BRIEF DESCRIPTION OF DRAWINGS

FIG. 1 shows that cardiac fibroblasts inhibit formation of vascular endothelial networks. FIG. 1A shows the protocol of the present example. FIG. 1B shows human dermal fibroblasts (NHDF) or cardiac fibroblasts (NHCF-a for cardiac atrium, and NHCF-v for cardiac ventricle) and human umbilical vein endothelial cells (HUVEC) were co-cultured and then immunostained using an anti-CD31 antibody. The green color shows CD 31-positive cells. FIG. 1C is a graph showing the total length of the vascular endothelial network shown in FIG. 1B. FIG. 1D is a graph showing the number of branch points of the vascular endothelial network shown in FIG. 1B.

FIG. 2 shows vascular endothelial networks obtained by co-culturing human dermal fibroblasts (NHDF) or human cardiac fibroblasts (NHCF-a for cardiac atrium, and NHCF-v for cardiac ventricle), and iPS cell-derived vascular endothelial cells (iPS-CD31+) or human heart-derived microvascular endothelial cells (HMVEC-C).

FIG. 3 shows that mouse cardiac fibroblasts inhibit formation of vascular endothelial networks. FIG. 3A shows the protocol of the present example. FIG. 3B shows cardiac muscle cells (green) and CD31-positive cells (red) obtained by co-culturing mouse dermal fibroblasts (DF) or mouse cardiac fibroblasts (CF), mouse ES cell-derived cardiac muscle cells, and mouse ES cell-derived vascular endothelial cells.

FIG. 4 shows that rat cardiac fibroblasts inhibit formation of vascular endothelial networks. FIG. 4A shows the protocol of the present example. FIG. 4B shows vascular endothelial networks obtained by co-culturing neonatal rat dermal fibroblasts (RDF) or neonatal rat cardiac fibroblasts (RCF), and neonatal rat heart-derived vascular endothelial cells. The green color represents CD31-positive cells, and the blue color (Hoechst 33342) represents nuclei. FIG. 4C is a graph showing the total length of the vascular endothelial network shown in FIG. 4B. FIG. 4D is a graph showing the number of branch points of the vascular endothelial network shown in FIG. 4B.

FIG. 5 compares the gene expressions of dermal fibroblasts and cardiac fibroblasts. FIG. 5A shows a heat map for glycoprotein-related genes. FIG. 5B shows a heat map for angiogenesis-related genes.

FIG. 6 shows the expression site of LYPD1. FIG. 6A is a graph showing the evaluation of relative expression levels of LYPD1 in various organs derived from rats by qPCR. FIG. 6B shows immunostained rat cardiac tissues (cTnT: cardiac troponin T (green), LYPD1 (red), DAPI: nuclei (blue), Merged: merged image).

FIG. 7 compares the LYPD1 gene expressions in human and rat primary cultured cells. FIG. 7A is a graph showing the evaluation of relative expression levels of LYPD1 in human primary dermal fibroblasts (NHDF) and human primary cardiac fibroblasts (cardiac atrium: NHCF-a, cardiac ventricle: NHCF-v) by qPCR. FIG. 7B is a graph showing the evaluation of relative expression levels of LYPD1 in rat primary dermal fibroblasts and rat primary cardiac fibroblasts by qPCR.

FIG. 8 shows that inhibition of LYPD1 (siRNA) rescues the vascular network formation. FIG. 8A shows the protocol of the present example. FIG. 8B shows human cardiac fibroblasts that have been transfected with an siRNA against LYPD1, co-cultured with HUVEC, and immunostained using an anti-CD31 antibody. The green color shows CD31-positive cells. FIG. 8C shows human cardiac fibroblasts that have been transfected with a control siRNA, co-cultured with HUVEC, and immunostained using an anti-CD31 antibody. The green color shows CD31-positive cells. FIG. 8D is a graph showing the total length of the vascular endothelial network shown in FIG. 8B and FIG. 8C.

FIG. 9 shows that inhibition of LYPD1 (anti-LYPD1 antibody) rescues the vascular network formation. FIG. 9A shows human cardiac fibroblasts and HUVEC were co-cultured and immunostained using an anti-CD31 antibody. The green color shows CD31-positive cells. FIG. 9B shows human cardiac fibroblasts and HUVEC co-cultured in the presence of an IgG control and then immunostained using an anti-CD31 antibody. The green color shows CD31-positive cells. FIG. 9C is a graph showing the total length of the vascular endothelial network shown in FIG. 9A and FIG. 9B. FIG. 9D is a graph showing the number of branch points of the vascular endothelial network shown in FIG. 9A and FIG. 9B.

FIG. 10 shows that inhibition of LYPD1 (anti-LYPD1 antibody) rescues the vascular network formation. FIG. 10A shows neonatal rat cardiac fibroblasts and neonatal rat heart-derived vascular endothelial cells were co-cultured in the presence of an anti-LYPD1 antibody and then immunostained using an anti-CD31 antibody. The green color shows CD31-positive cells. FIG. 10B shows neonatal rat cardiac fibroblasts and neonatal rat heart-derived vascular endothelial cells were co-cultured in the presence of an IgG control and then immunostained using an anti-CD31 antibody. The green color shows CD31-positive cells. FIG. 10C is a graph showing the total length of the vascular endothelial network shown in FIG. 10A and FIG. 10B, FIG. 10D is a graph showing the number of branch points of the vascular endothelial network shown in FIG. 10A and FIG. 10B.

FIG. 11 shows the results of microarray analysis for the gene expression in human dermoblasts (NHDF), human cardiac fibroblasts (NHCF), iPS-derived stromal cells, and mesenchymal stem cells (MSC). Cluster analysis is shown on the right.

FIG. 12 shows that human iPS-derived stromal cells (iPS fibro-like) inhibit formation of vascular endothelial networks from human iPS CD31-positive cells (iPS CD31+). FIG. 12A shows the protocol of the present example. FIG. 12B shows human dermal fibroblasts (NHDF) or human iPS-derived stromal cells were co-cultured with human iPS CD31-positive cells and then immunostained using an anti-CD31 antibody. The red color shows CD31-positive cells. FIG. 12C is a graph showing the evaluation of the LYDP1 expression in human dermal fibroblasts (NHDF), human cardiac fibroblasts (NHCFa), and human iPS-derived stromal cells (iPS fibro-like) by qPCR.

FIG. 13 shows that a recombinant LYPD1 inhibits formation of vascular endothelial networks. FIG. 13A shows detection of a FLAG-LYPD1 protein by using a peroxidase-conjugated anti-DYKDDDDK tag monoclonal antibody (top) or a rabbit anti-LYPD1 polyclonal antibody (bottom), in which the protein has been purified using an anti-DYKDDDDK tag antibody-magnetic beads and subjected to sodium dodecyl sulfate-polyacrylamide gel electrophoresis or immunoblotting. FIG. 13B shows the appearance of the vascular endothelial network (tube) formation after treatment with a recombinant LYPD1 protein. CD31 (green) and nuclei (Hoechst 33342 (blue)) have been stained. The scale bar represents 400 μm. FIG. 13C shows the total length of the vascular endothelial network (tube) after treatment with a recombinant LYPD1 protein. The total length has been calculated by summing the length of the tubes formed by CD31-positive cells. Values have been calculated as the mean±standard deviation from three independent experiments. P<0.05.

FIG. 14 shows that the inhibitory effect of cardiac fibroblasts on angiogenesis does not depend on the number of vascular endothelial cells. FIG. 14A shows human cardiac atrium-derived fibroblasts (NHCF-a) (2×10⁴ cells/cm², 4×10⁴ cells/cm², and 6×10⁴ cells/cm²) and human umbilical vein endothelial cells (HUVEC) (2.4×10⁵ cells/cm²) were co-cultured and then stained with an anti-CD31 antibody and Hoechst. The green color shows CD31-positive cells, while the blue color shows nuclei. FIG. 14B is a graph showing the total length of the vascular endothelial network shown in FIG. 14A. Neither number of cells has shown a significant difference.

FIG. 15 shows an inhibitory effect of recombinant LYPD1 on the formation of vascular endothelial networks in a Matrigel® tube formation assay. HUVECs (1.0×10⁴ cells/cm²) have been plated on wells in a 96-well plate pre-coated with Matrigel® and cultured in the absence (control) or presence (1 μg/mL, 2 μg/mL, or 5 μg/mL) of a recombinant LYPD1 protein for 20 hours (5% CO₂, 37° C.). The scale bar represents 500 μm.

### DESCRIPTION OF EMBODIMENTS

In the process of studying the construction of tissue-engineered three-dimensional biological tissues, the inventors have found that co-culturing of cardiac fibroblasts and vascular endothelial cells that are derived from any mammal (mouse, rat or human) results in significant inhibition of the formation of vascular endothelial cell networks. The inventors have investigated the cause in detail and found that an LYPD1 protein is involved in the inhibition of the formation of vascular endothelial networks. The present invention has been completed based on these findings.

1. Formation of Vascular Endothelial Networks (Angiogenesis)

As used herein, the term “vascular endothelial network” means a capillary-like network constructed in biological tissues by vascular endothelial cells and/or vascular endothelial precursor cells. CD31, known as a cell surface marker protein of vascular endothelial cells and/or vascular endothelial precursor cells, can be detected by any method to find vascular endothelial cells and/or vascular endothelial precursor cells in biological tissues. Vascular endothelial cells and/or vascular endothelial precursor cells arrange into luminal structures to form vascular networks through which fluids, especially blood, pass. Biological tissues need a blood supply containing nutrients and oxygen throughout their bodies to survive. This requires densely constructed vascular networks. However, excessive formation of vascular endothelial networks can cause or increase the severity of angiogenesis-related diseases (described later). Whether the formation of vascular endothelial networks (angiogenesis) is inhibited can be determined by evaluating the length and/or branch points of vascular endothelial networks formed as described above. The length of vascular endothelial networks means the total length of vascular endothelial networks per unit area. The branch points of vascular endothelial networks mean the total number of junctions among vascular endothelial networks per unit area. In a method for screening angiogenesis inhibitors as described later, as the length and/or branch points of vascular endothelial networks are smaller than the case without a test substance (or with a negative control compound), the ability of the angiogenesis inhibitor to inhibit formation of vascular endothelial networks can be evaluated as being higher. The length and/or branch points of vascular endothelial networks can be determined by using images obtained with a confocal fluorescence microscope or the like and considering the CD31-positive regions as vascular endothelial cells, for example, using MetaXpress software (Molecular Devices, LLC).

2. Angiogenesis Inhibitor

As used herein, the term “angiogenesis inhibitors” refers to LYPD1 protein, or derivatives thereof, or parts thereof, or vectors for expressing the same, or cells expressing the same, or naturally occurring or synthesized compounds or cells that directly and/or indirectly increase the expression of LYPD1 protein and inhibit formation of vascular endothelial networks (angiogenesis). Angiogenesis inhibitors can also be obtained by a method for screening angiogenesis inhibitors that increase the expression of LYPD1 protein as described later. In one embodiment, the angiogenesis inhibitor of the present invention may be a pharmaceutically acceptable salt thereof. As used herein, the term “pharmaceutical” or “pharmaceutically acceptable” means molecules and compositions that do not cause side effects, allergic reactions, or other adverse effects when properly administered to mammals, especially human. As used herein, pharmaceutically acceptable carriers or vehicles means non-toxic solid, semi-solid, or liquid injections, diluents, encapsulating agents, or any kind of formulation aids. Pharmaceutically acceptable carriers or vehicles can be used with the angiogenesis inhibitor of the present invention.

2-1. LYPD1 Protein

As used herein, the term “LYPD1 protein” refers to a protein having the same meaning as that commonly used in the art and also called as LY6/PLAUR domain containing 1, PHTS, LYPDC1 (hereinafter, also referred to as “LYPD1”). LYPD1 protein is widely conserved and has been found, for example, in humans, monkeys, dogs, cattle, mice, rats. mRNA and amino acid sequences of naturally occurring human LYPD1 are deposited, for example, in GenBank database and GenPept database under Accession Nos. NM_001077427 (SEQ ID NO: 1) and NP_001070895 (SEQ ID NO: 2), NM_144586 (SEQ ID NO: 3) and NP_653187 (SEQ ID NO: 4), NM_001321234 (SEQ ID NO: 5) and NP_001308163 (SEQ ID NO: 6), and NM_001321235 (SEQ ID NO: 7) and NP_001308164 (SEQ ID NO: 8), and in UniProt KB database, under Accession No. Q8N2G4-2 (SEQ ID NO: 19). mRNA and amino acid sequences of naturally occurring mouse LYPD1 are deposited, for example, in GenBank database and GenPept database under Accession Nos. NM_145100 (SEQ ID NO: 9) and NP_659568 (SEQ ID NO: 10), NM_001311089 (SEQ ID NO: 11) and NP_001298018 (SEQ ID NO: 12), and NM_001311090 (SEQ ID NO: 13) and NP_001298019 (SEQ ID NO: 14).

LYPD1 protein is known to be highly expressed in brain, but little is known so far about its function. Based on the amino acid motif, LYPD1 protein is thought to be a glycosylphosphatidylinositol (GPI)-anchored protein.

As used herein, the term “LYPD1 protein” refers to naturally occurring LYPD1 protein, or mutants and variants thereof (collectively referred to as “derivatives”), or parts thereof. This term may also mean a fusion protein obtained by fusing a domain of LYPD1 protein keeping at least one LYPD1 activity with, for example, another peptide. LYPD1 protein may be derived from any organisms, preferably from mammals (e.g., human, non-human primates, rodents (such as mice, rats, hamsters, and guinea pigs), rabbits, dogs, cattle, horses, pigs, cats, goats, sheep), more preferably from human or non-human primates, particularly preferably from human. LYPD1 protein used in the present invention is a protein having a sequence selected from SEQ ID NOs: 1 to 14 and 19, or having a sequence identity with a sequence selected from SEQ ID NOs: 1 to 14 and 19 of at least 85% or more, preferably 90% or more, more preferably 95% or more, still more preferably 97% or more, most preferably 99% or more.

LYPD1 protein of the present invention may be a protein encoded in DNA that hybridizes under stringent conditions with a probe that can be prepared from the nucleotide sequence of the LYPD1 protein gene described above, e.g., a complementary sequence to the whole or part of said nucleotide sequence, provided that the protein maintains the original function. Such a probe can be prepared, for example, by PCR using oligonucleotides prepared based on the nucleotide sequence as primers and a DNA fragment comprising the nucleotide sequence as a template. The term “stringent conditions” refers to those conditions that permits so-called specific hybridization but not non-specific hybridization. For example, the term may include those conditions that permits hybridization between DNAs with high homology, for example, DNAs with a homology of 80% or more, preferably 90% or more, more preferably 95% or more, still more preferably 97% or more, particularly preferably 99% or more, but not hybridization between DNAs with a lower homology, or washing conditions at salt concentrations and temperature equivalent to those in normal Southern hybridization, at 60° C. in 1×SSC and 0.1% SDS, preferably at 60° C. in 0.1×SSC and 0.1% SDS, more preferably at 68° C. in 0.1×SSC and 0.1% SDS, once, preferably two or three times. For example, when a DNA fragment of about 300 bp in length is used as a probe, washing conditions in hybridization may be at 50° C. in 2×SSC and 0.1% SDS.

For the method of obtaining LYPD1 protein, it can be obtained by using, for example, known genetic and protein engineering techniques, for example, by introducing into any cells a vector designed to express an artificial 8 amino acid sequence called FLAG tag (DYKDDDDK, Asp-Tyr-Lys-Asp-Asp-Asp-Asp-Lys) at N or C terminus of LYPD1 protein, culturing the cells, and purifying the expressed proteins with an anti-FLAG tag antibody-conjugated resin. Alternatively, other tags (e.g., BCCP, c-myc tag, calmodulin tag, HA tag, His tag, maltose binding protein tag, Nus tag, glutathione-S-transferase (GST) tag, green fluorescent protein tag, thioredoxin tag, S tag, Strep-tag II, Softag1, Softag3, T7 tag, elastin-like peptides, chitin-binding domains, and xylanase 10A, etc.) -fused LYPD protein can be expressed in any cells and purified by an optimal method selected based on the tag. Without using tags, cells expressing LYPD1 protein may be homogenized and directly purified using, for example, anti-LYPD1 antibodies.

LYPD1 protein can be obtained through expression using, for example, plant cells, Escherichia coli, yeasts, insect cells, animal cells, or extracts thereof, preferably insect cells or mammal cells, more preferably mammal cells.

2-2. LYPD1 Protein Expression Vector

In one embodiment of the present invention, LYPD1 protein, or a derivative thereof, or a part thereof as an angiogenesis inhibitor may be expressed from an expression vector obtained by incorporating a nucleic acid encoding it into any vector (hereinafter, collectively referred to as “LYPD1 protein expression vectors”). In the present invention, any known vectors may be selected as appropriate for use in the LYPD1 protein expression vector. Examples include plasmid vectors, cosmid vectors, fosmid vectors, virus vectors, and artificial chromosome vectors. A nucleic acid encoding LYPD1 protein, or a derivative thereof, or a part thereof is introduced into any vector by any method known in the art.

2-3. LYPD1 Protein-Expressing Cell

In one embodiment of the present invention, LYPD1 protein, or a derivative thereof, or a part thereof as an angiogenesis inhibitor may be expressed from any cells (hereinafter, referred to as “LYPD1 protein-expressing cells”). For example, LYPD1 protein-expressing cells may be transfected with the LYPD1 protein expression vector described above. The LYPD1 protein expression vector may be introduced into cells according to any methods known in the art. Cells having the LYPD1 protein expression vector introduced therein and transiently or constitutively expressing LYPD1 protein may be selected by any method, including a selection method by using drugs corresponding to the drug resistance genes encoded in the expression vector (e.g., neomycin, hygromycin, etc.). Cells that can be used for transfection may be isolated from living bodies, preferably from a subject to be treated. Cells derived from a subject to be treated are less likely to be rejected by the immune system when administered to the subject.

In one embodiment of the present invention, LYPD1 protein-expressing cells may be those isolated from living bodies, including cells that highly express LYPD1 protein as compared to skin-derived fibroblasts, preferably stromal cells or fibroblasts found in biological tissues in brain, heart, kidney, or muscle, more preferably fibroblasts derived from heart.

In one embodiment of the present invention, the LYPD1 protein-expressing cells to be used can be those with their expression of the LYPD1 gene directly and/or indirectly increased by genome editing techniques. As used herein, genome editing nucleic acids refer to nucleic acids used for editing desired genes in a system using nucleases used in gene targeting. The nucleases used in gene targeting include known nucleases, as well as new nucleases used for gene targeting in future. Examples of the known nucleases include CRISPR/Cas9 (Ran, F. A., et al., Cell, 2013, 154, 1380-1389), TALEN (Mahfouz, M., et al., PNAS, 2011, 108, 2623-2628), and ZFN (Urnov, F., et al., Nature, 2005, 435, 646-651). Genome editing techniques can induce mutations, for example, in the promoter region and/or enhancer region in the LYPD1 gene. This will successfully provide cells that highly express LYPD1 protein.

The LYPD1 protein-expressing cells obtained by genome editing techniques are preferably cells highly expressing LYPD1 protein as compared to skin-derived fibroblasts, more preferably cells expressing LYPD1 protein at the same or higher level as heart-derived fibroblasts (for example, cells expressing 80% or more, 90% or more, 100% or more, 110% or more, 120% or more, 130% or more, 140% or more, 150% or more, 160% or more, 170% or more, 180% or more, 190% or more, 200% or more of LYPD1 protein as compared with the expression level of LYPD1 protein expressed in heart-derived human fibroblasts).

In one embodiment of the present invention, the LYPD1 protein-expressing cells may be derived from pluripotent stem cells. As used herein, pluripotent stem cells refer to cells that have self-renewal and multi-differentiation capabilities and are capable of forming any cell that makes up the body (pluripotent). The self-renewal capability means the ability of a single cell to produce two undifferentiated cells identical to itself. Pluripotent stem cells used in the present invention include embryonic stem cells (ES cells), embryonic carcinoma cells (EC cells), trophoblast stem cells (TS cells), epiblast stem cells (EpiS cells), embryonic germ cells (EG cells), multipotent germline stem cells (mGS cells), and induced pluripotent stem cells (iPS cells). Differentiation of such pluripotent stem cells can be induced according to, for example, a method by Matsuura et al. (Matsuura K., et al., Creation of human cardiac cell sheets using pluripotent stem cells. Biochem. Biophys. Res. Commun., 2012 Aug. 24; 425 (2): 321-327).

2-4. Angiogenesis Inhibitor Compounds that Increase the Expression of LYPD1 Protein

In the present specification, angiogenesis inhibitor compounds that increase the expression of LYPD1 protein may include low-molecular weight organic small molecules, peptides, proteins, tissue extracts or cell culture supernatants from mammals (e.g., mice, rats, pigs, cattle, sheep, monkeys, human), plant-derived compounds or extracts (e.g., crude drug extracts, crude drug-derived compounds), and microorganism-derived compounds or extracts or culturing products. In the present invention, the angiogenesis inhibitor compounds that increase the expression of LYPD1 protein refer to those that exert a direct and/or indirect influence, increase the expression of LYPD1, and inhibit formation of vascular endothelial networks (angiogenesis), and can be selected from test substances by a screening method described later.

3. Pharmaceutical Composition

The present invention provides a pharmaceutical composition for use in treatment or prevention of angiogenesis-related diseases, comprising as an active ingredient an angiogenesis inhibitor, especially LYPD1 protein, or a derivative thereof, or a part thereof, or a vector for expressing the same, or a cell expressing the same, or a naturally occurring or synthesized compound or a cell that directly and/or indirectly increases the expression of LYPD1 protein and inhibits the formation of vascular endothelial networks (angiogenesis). The pharmaceutical composition of the present invention can be administered to a subject in need thereof to treat or prevent angiogenesis-related diseases. The pharmaceutical composition of the present invention may comprise a pharmaceutically acceptable carrier or vehicle.

4. Administration of the Angiogenesis Inhibitor or Pharmaceutical Composition to a Subject

The angiogenesis inhibitor or the pharmaceutical composition according to the present invention is administered to a subject in a therapeutically effective amount. The term “therapeutically effective amount” means a necessary and sufficient amount of an angiogenesis inhibitor to provide a desired inhibitory effect on angiogenesis.

As used herein, the term “administration” means providing a predetermined substance to a subject in any suitable method. As for the route of administration, the angiogenesis inhibitor or the pharmaceutical composition of the present invention can be orally or parenterally administered via any general route, provided that it can be delivered to a target tissue. The angiogenesis inhibitor or the pharmaceutical composition of the present invention can also be administered using any device that delivers active ingredients to target cells.

As used herein, the term “subject” refers to animals including, but are not limited to, human, non-human primates, rodents (such as mice, rats, hamsters, guinea pigs), rabbits, dogs, cattle, horses, pigs, cats, goats, and sheep, etc. In one embodiment, the subject represents a mammal. In other embodiments, the subject represents human.

The amount of the angiogenesis inhibitor of the present invention used per day is determined within the medical judgment of the physician. The therapeutically effective amount varies depending on the disorder to be treated and/or prevented and the severity of the disorder, the activity of the compound to be used, the composition to be used, the age and weight of the patient, the health status, sex, and diet of the patient, the tuning and route of administration, the clearance of, treatment duration of, and drugs in combination with the compound to be used, as well as other factors well-known in the medical arts. For example, those skilled in the art can start the administration of the angiogenesis inhibitor at a lower dosage than required to achieve the desired therapeutic effect and then gradually increase the dosage until the desired effect is achieved within the skill of the art. The dose of the angiogenesis inhibitor can broadly vary from 0.01 to 1,000 mg per day for adults. The pharmaceutical composition comprising the angiogenesis inhibitor as an active ingredient contains 0.01, 0.05, 0.1, 0.5, 1.0, 2.5, 5.0, 10.0, 15.0, 25.0, 50.0, 100, 250, or 500 mg of the active ingredient for administration customized to the symptoms in the patient to be treated. The pharmaceutical composition usually contains about 0.01 mg to about 500 mg of the active ingredient, preferably 1 mg to about 100 mg of the active ingredient. The effective dose of the drug usually ranges from 0.0002 mg/kg body weight/day to about 20 mg/kg body weight/day. In particular, the drug is administered at a dose from about 0.001 mg/kg body weight/day to 7 mg/kg body weight'day.

When the angiogenesis inhibitor or the pharmaceutical composition comprises a cell expressing LYPD1 protein, or a derivative thereof, or a part thereof, the therapeutically effective dose (number of cells) varies depending on the form or expression level of the LYPD1 protein, or derivative thereof, or part thereof.

In one embodiment of the present invention, when the angiogenesis inhibitor or the pharmaceutical composition comprises a cell expressing LYPD1 protein, or a derivative thereof, or a part thereof, a suspension containing the angiogenesis inhibitor or the pharmaceutical composition may be injected to or around the affected area. Alternatively, a “biological tissue” comprising the angiogenesis inhibitor or the pharmaceutical composition may be prepared and administered (transplanted) to a subject. Biological tissues would be able to adhere to an affected area, and continuously release the angiogenesis inhibitor around the affected area, thereby sustaining the inhibitory effect on angiogenesis.

“Biological tissues” can be prepared using known methods. For example, biological tissues obtained by methods of preparing biological tissues by laminating cell sheets on a vascular bed (see WO2012/036224 and WO2012/036225), methods of preparing biological tissues by 3D printing technology (see WO2012/058278), methods of preparing three-dimensional structures using cells covered with an adhesive layer (see Japanese Unexamined Patent Application No. 2012-115254), and methods of preparing organs in vivo (see Kobayashi T., Nakauchi H. [From cell therapy to organ regeneration therapy: generation of functional organs from pluripotent stein cells]. Nihon Rinsho. 2011 December; 69 (12): 2148-55; WO2010/021390; WO2010/097459), as well as known production methods can be applied in the present invention and fall within the scope of the present invention.

As used herein, the term “cell sheet” refers to a cell population in the form of a single- or multi-layered sheet obtained by culturing a cell population comprising a plurality of any cells on a cell culture substrate, and detaching it from the cell culture substrate. Methods for obtaining cell sheets include, for example, a method comprising culturing cells on a stimuli-responsive culture substrate covered with polymers that change its molecular structure in response to stimuli such as temperature, pH, and light, changing the stimulus conditions such as temperature, pH, and light to change the surface of the stimuli-responsive culture substrate, and detaching the sheet-forming cells from the stimuli-responsive culture substrate while maintaining the cell-to-cell adhesion; and a method comprising culturing cells on any culture substrate, and detaching it by physical means such as using tweezers. Known stimuli-responsive culture substrates for obtaining cell sheets include a temperature-responsive culture substrate, the surface of which is covered with a polymer that exhibits varying hydration capacity in a temperature range of 0-80° C. After cells are cultured on the temperature-responsive culture substrate in a temperature range in which the polymer exhibits low hydration capacity, the cells can be detached and recovered in a form of sheet by changing the temperature of the culture medium to a temperature at which the polymer exhibits high hydration capacity.

The temperature-responsive culture substrate used to obtain the cell sheet is preferably a substrate that changes the hydration capacity of its surface in a temperature range at which the cells can be cultured. Preferred temperature range is typically temperatures at which cells are cultured, for example, 33° C. to 40° C. The temperature-responsive polymer to be coated on the culture substrate used to obtain the cell sheet may be either a homopolymer or a copolymer. Examples of such a polymer include polymers described in Japanese Unexamined Patent Application No. H2-211865.

For example, a temperature-responsive culture dish employing a stimuli-responsive polymer, especially a temperature-responsive polymer, poly(N-isopropylacrylamide), will be described. Poly(N-isopropylacrylamide) is known to be a polymer having a lower critical solution temperature of 31° C. The polymer, when in a free state, is dehydrated in water at a temperature of 31° C. or higher, so that the polymer chains are aggregated and cause cloudiness. Conversely, at a temperature of lower than 31° C., the polymer chains are hydrated and dissolved in water. In the present invention, this polymer is coated and fixed on the surface of a substrate such as dish. Thus, the polymer on the surface of the culture substrate is dehydrated at a temperature of 31° C. or higher as described above. Since the polymer chains are fixed on the surface of the culture substrate, the surface of the culture substrate will exhibit hydrophobic properties. Conversely, the polymer on the surface of the culture substrate is hydrated at a temperature of lower than 31° C. Since the polymer chains are coated on the surface of the culture substrate, the surface of the culture substrate will exhibit hydrophilic properties. The hydrophobic surface in this situation allows cells to adhere thereto and grow thereon, while the hydrophilic surface does not allow cells to adhere thereto. Thus, when the substrate is cooled to lower than 31° C., cells are detached from the surface of the substrate. Cells cultured to confluence over the culture surface can be recovered as a cell sheet by cooling the substrate to lower than 31° C. Any temperature-responsive culture substrates having the same function can be used, including UpCell® commercially available from CellSeed Inc. (Tokyo, Japan).

The biological tissue used in one embodiment of the present invention may be a layered cell sheet obtained by layering a plurality of cell sheets. Examples of the method for preparing the layered cell sheet include a method comprising aspirating a cell sheet floating in a culture medium together with the culture medium using a pipette or the like, releasing the cell sheet onto a cell sheet on another culture dish, and finishing the layering of the cell sheet using liquid flow, and a layering method employing a tool for transferring cells. Other known methods can be used to obtain a biological tissue comprising layered cell sheets.

The angiogenesis inhibitor or the pharmaceutical composition comprising, as an active ingredient, LYPD1 protein, or a derivative thereof, or a part thereof, or a vector for expressing the same, or a cell expressing the same, or a naturally occurring or synthesized compound or a cell that directly and/or indirectly increases the expression of LYPD1 protein and inhibits the formation of vascular endothelial networks (angiogenesis) of the present invention allows for inhibiting the angiogenesis. Examples of angiogenesis-related diseases that can be treated or prevented include solid cancer, diabetic retinopathy, age-related macular degeneration, retinopathy of prematurity, corneal graft rejection, neovascular glaucoma, erythroderma, proliferative retinopathy, psoriasis, hemophilic arthropathy, capillary proliferation in atherosclerotic plaques, keloid, wound granulation, vascular adhesion, rheumatoid arthritis, osteoarthritis, autoimmune diseases, Crohn's disease, restenosis, atherosclerosis, intestinal adhesion, ulcer, liver cirrhosis, glomerulonephritis, diabetic nephropathy, malignant nephrosclerosis, thrombotic microangiopathy, organ graft rejection, glomerulopathy, diabetes mellitus, inflammation, and neurodegenerative diseases. The angiogenesis inhibitor or pharmaceutical composition of the present invention can be used to inhibit abnormal angiogenesis, allowing for treatment or prevention of the above-described diseases.

Examples of solid cancers that can be treated or prevented by using the angiogenesis inhibitor or the pharmaceutical composition comprising as an active ingredient LYPD1 protein, or a derivative thereof, or a part thereof, or a vector for expressing the same, or a cell expressing the same of the present invention include cervical cancer, lung cancer, pancreatic cancer, non-small-cell lung cancer, liver cancer, colon cancer, osteosarcoma, skin cancer, head cancer, neck cancer, cutaneous melanoma, intraocular melanoma, uterine cancer, ovarian cancer, rectal cancer, liver cancer, brain tumor, bladder cancer, gastric cancer, perianal gland cancer, colon cancer, breast cancer, fallopian tube cancer, endometrial cancer, vaginal cancer, vulvar cancer, Hodgkin's lymphoma, esophageal cancer, small intestine cancer, endocrine cancer, thyroid cancer, parathyroid cancer, adrenal cancer, soft tissue sarcoma, urethral cancer, penile cancer, prostate cancer, bladder cancer, kidney cancer, ureter cancer, renal cell cancer, renal pelvic cancer, central nervous system (CNS) tumor, primary CNS lymphoma, spinal cord tumor, brainstem glioma, and pituitary adenoma. The angiogenesis inhibitor or the pharmaceutical composition of the present invention can be used to inhibit angiogenesis around the above-described solid cancers and deplete nutrients and oxygen required for proliferation and growth, allowing for treatment or prevention of the above-described solid cancers. The angiogenesis inhibitor or the pharmaceutical composition of the present invention also prevents metastasis of the above-described solid cancers. Particularly in the treatment of solid cancers, the angiogenesis inhibitor of the present invention that inhibits angiogenesis allowing for supply of nutrients to tumor without directly affecting cancer cells can also advantageously avoid drug resistance in cancer cells.

In one embodiment, the angiogenesis inhibitor or the pharmaceutical composition comprising, as an active ingredient, LYPD1 protein, a derivative thereof, a part thereof, a vector for expressing the same, or a cell expressing the same of the present invention may further comprise a known anticancer agent or angiogenesis inhibitor, and can be used in combination with other known therapies for use in treatment of the above-described diseases. Examples of the other therapies include, but not limited to, chemotherapy, radiation therapy, hormone therapy, bone marrow transplantation, stein cell therapy, other biological therapies, and immunotherapy.

Examples of other anticancer agents that may be contained in the angiogenesis inhibitor or the pharmaceutical composition of the present invention include, but not limited to, DNA alkylating agents (such as mechlorethamine, chlorambucil, phenylalanine, cyclophosphamide, ifosfamide, carmustine, lomustine, streptozotocin, busulfan, thiotepa, cisplatin, and carboplatin), anticancer antibiotics (such as actinomycin D, doxorubicin, daunorubicin, idarubicin, mitoxantrone, plicamycin, mitomycin C, bleomycin), and plant alkaloids (such as vincristine, vinblastine, paclitaxel, docetaxel, etoposide, teniposide, topotecan, and irinotecan).

Examples of other angiogenesis inhibitors that may be contained in the angiogenesis inhibitor or the pharmaceutical composition of the present invention include, but not limited to, Angiostatin, Antiangiogenic antithrombin III, Angiozyme, ABT-627, Bay 12-9566, Benefin, Bevacizumab, BMS-275291, Cartilage-derived inhibitors, CAI, CD59 complement fragment, CEP-7055, Col 3, Combretastatin A-4, Endostatin (type XVIII collagen fragment), Fibronectin fragment, Gro-β, Halofuginone, Heparinase, Heparin hexasaccharide fragment, HMV833, Human chorionicgonadotropin (hCG), IM-862, Interferon alpha/beta/gamma, Interferon inducible protein (IP-10), Interleukin-12, Kringle 5 (plasminogen fragment), Marimastat, Dexamethasone, Metalloprotease inhibitors (TIMP), 2-Methoxyestradiol, MMI1270 (CGS 27023A), MoAb IMC-1C11, Neovastat, NM-3, Panzem, PI-88, Placenta ribonuclease inhibitors, Plasminogen activator inhibitors, Platelet factor 4 (PF4), Prinomastat, Prolactin 16 kD fragment, Proliferin-related proteins (PRP), PTK 787/ZK 222594, Retinoids, Solimastat, squalamine, SS 3304, SU 5416, SU6668, SU11248, Tetrahydrocortisol-S, Tetrathiomolybdate, Thalidomide, Thrombospondin-1 (TSP-1), TNP-470, Transforming growth factor-β (TGF-β), Vasculostatin, Vasostatin, ZD6126, ZD6474, Farnesyltransferase inhibitors (FTI), and Bisphosphonates (e.g., alendronate, etidronate, pamidronate, risedronate, ibandronate, zoledronate, olpadronate, incadronate, and neridronate).

5. Use of Angiogenesis Inhibitor for Production of Pharmaceutical Compositions

In one embodiment, the angiogenesis inhibitor of the present invention can be used to produce a pharmaceutical composition for treatment or prevention of angiogenesis-related diseases.

6. Method for Screening Angiogenesis Inhibitors

The angiogenesis inhibitor of the present invention can be identified from candidate substances (test substances) by applying a known screening method. For example, a method comprising the following steps may be used:

a step (i) of treating and culturing a first cell with a test substance; and

a step (ii) of detecting the expression level of LYPD1 protein in the first cell and comparing it with that of an untreated first cell.

The expression level of LYPD1 protein may be detected using known methods. For example, the expression level can be evaluated using well known techniques, such as quantitative PCR (qPCR), Western blotting, flow cytometry (FACS), ELISA, and immunohistochemistry.

The first cell may be a cell that poorly expresses LYPD1 protein, for example, a skin-derived, esophagus-derived, testis-derived, lung-derived, or liver-derived cell, preferably a skin-derived, esophagus-derived, testis-derived, lung-derived, or liver-derived fibroblast, more preferably a skin-derived fibroblast.

In one embodiment, the method for screening angiogenesis inhibitors of the present invention can further comprise the following steps:

a step (iii) of selecting a test substance that enhance the expression of LYPD1 protein as compared with the level of LYPD1 protein in the untreated first cell in the step (ii);

a step (iv) of adding the test substance to a cell population comprising a second cell and a vascular endothelial cell and/or precursor cell, and culturing the cell population; and

a step (v) of detecting vascular endothelial networks formed by the vascular endothelial cell and/or precursor cell.

For the second cell, cells poorly expressing LYPD1 (2.4×10⁵ cells/cm²), vascular endothelial cells and/or vascular endothelial precursor cells that form vascular networks (for example, 2.0×10⁴ cells/cm²), and the test substance selected in the step (iii) above are pre-incubated and then plated on a culture dish and cultured at 37° C., 5% CO₂ for several days. Thereafter, vascular endothelial networks formed by the vascular endothelial cells and/or vascular endothelial precursor cells can be observed by microscopy (preferably fluorescence microscopy) to evaluate the length of and the number of branch points in the vascular endothelial networks.

The second cell may be a cell that poorly expresses LYPD1 protein, for example, a skin-derived, esophagus-derived, testis-derived, lung-derived, or liver-derived cell, preferably a skin-derived, esophagus-derived, testis-derived, lung-derived, or liver-derived fibroblast, more preferably a skin-derived fibroblast.

Vascular endothelial networks formed by vascular endothelial cells and/or vascular endothelial precursor cells may be evaluated by detecting it with a fluorescence-labeled anti-CD31 antibody or vascular endothelial cell-specific antibody. Alternatively, evaluation may be made, for example, by using vascular endothelial cells and/or vascular endothelial precursor cells expressing a fluorescent protein such as GFP and detecting the fluorescence.

### EXAMPLES

The present invention will be described in more detail with reference to Examples which by no means limit the scope of the present invention.

<Cells Used and Preparation Methods>

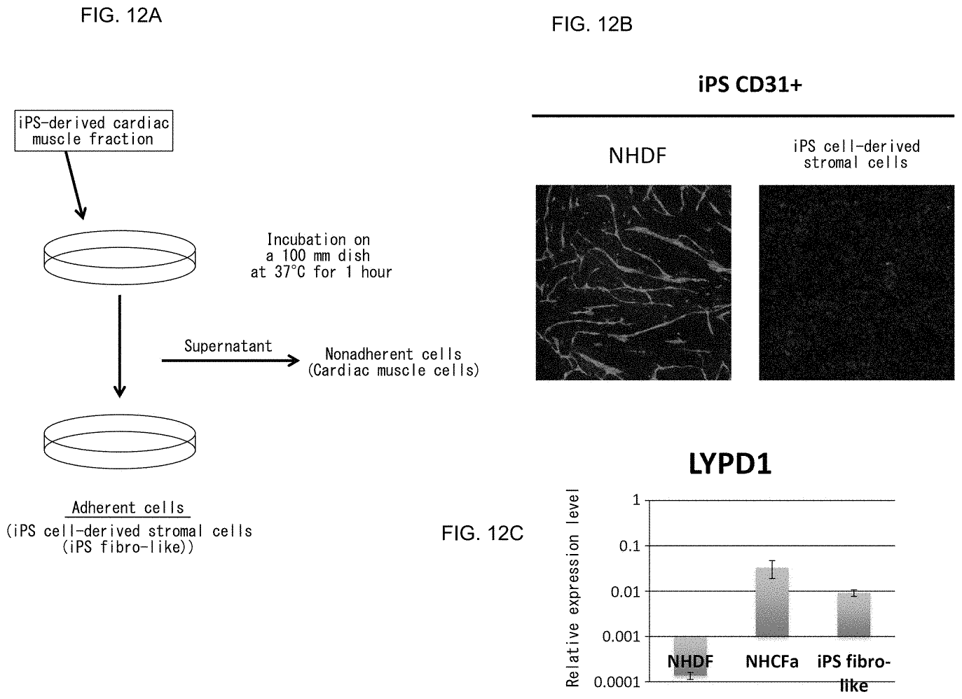

Cells that were used in the following Examples were as follows:
Human dermal fibroblast (purchased from Lonza, NHDF-Ad, normal human dermal fibroblast (CC-2511))
Human cardiac fibroblast (purchased from Lonza, NHCF-a (normal human cardiac fibroblast-cardiac atrium (CC-2903)), NHCF-v (normal human cardiac fibroblast-cardiac ventricle (CC-2904))
Human umbilical vein endothelial cell (HUVEC) (purchased from Lonza, Cat. #C2517A))
Normal human cardiac microvascular endothelial cell (HMVEC-C) (purchased from Lonza, Cat. #CC-7030)
Human iPS-derived stromal cell: a cell population obtained during differentiation of human iPS cells into cardiac muscle cells are sorted for a cell population with higher adhesion to a culture dish than cardiac muscle cells, to obtain fibroblast-like cells. The fibroblast-like cells were considered as human iPS-derived stromal cells (see FIG. 12(A)). Differentiation of human iPS cells into cardiac muscle cells was performed according to a method described in Matsuura K., et al. Creation of human cardiac cell sheets using pluripotent stem cells. Biochem Biophys Res Commun. 2012 Aug. 24; 425 (2): 321-7.
Human iPS cell-derived vascular endothelial cells (iPS-CD31+) were obtained through preparation with reference to the following (White M P., et al., Stem Cells. 2013 January; 31 (1): 92-103).
Cos-7 cells (from National Institutes of Biomedical Innovation, Health and Nutrition JCRB Cell Bank)

### Example 1

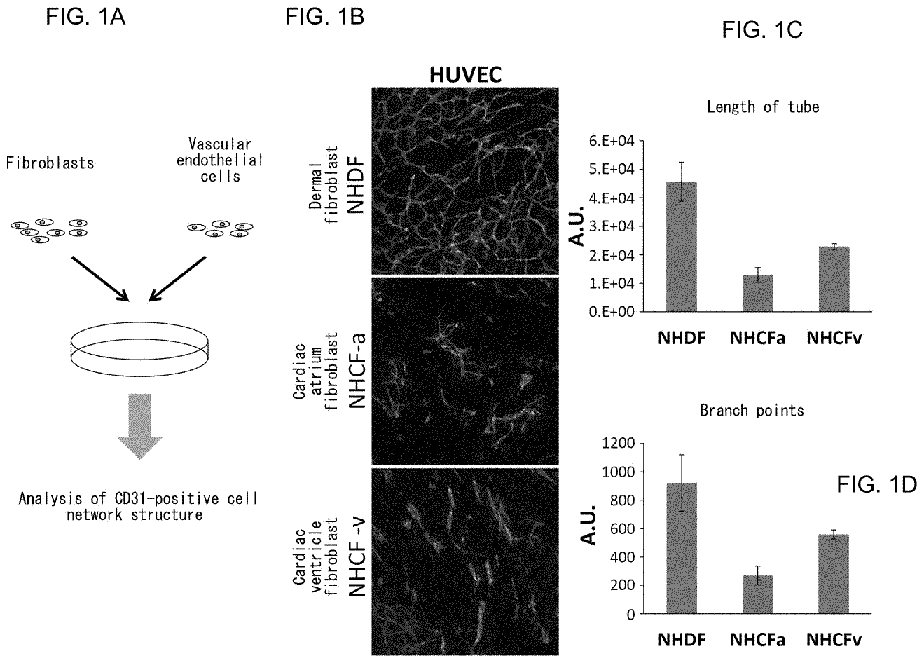

Cardiac Fibroblasts Inhibit Formation of Vascular Endothelial Networks (FIG. 1)

Human dermal fibroblasts (NHDF) or cardiac fibroblasts (cardiac atrium-derived: NHCF-a, cardiac ventricle-derived: NHCF-v) (2.4×10⁵ cells/cm²), and human umbilical vein endothelial cells (HUVEC) (2.0×10⁴ cells/cm²) were co-cultured for 3 days at 5% CO₂, 37° C. and then immunostained with an anti-CD31 antibody (Human CD31/PECAM-1 PE-conjugated Antibody, FAB3567P, R&D Systems, Inc.). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined.

Formation of vascular endothelial networks was promoted in co-culture with human dermal fibroblasts, but inhibited in co-culture with human cardiac fibroblasts.

### Example 2

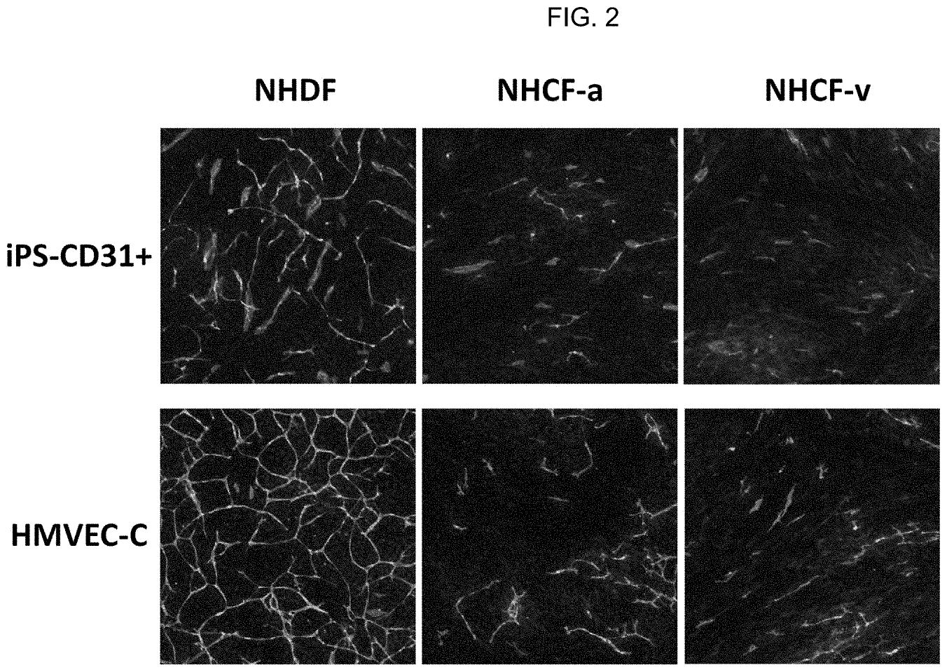

Cardiac Fibroblasts Inhibit Formation of Vascular Endothelial Networks (FIG. 2)

Human dermal fibroblasts or cardiac fibroblasts (2.4×10⁵ cells/cm²), and iPS cell-derived vascular endothelial cells (iPS-CD31+) or normal human heart microvascular endothelial cells (HMVEC-C) (2.0×10⁴ cells/cm²) were co-cultured for 3 days at 5% CO₂, 37° C. and then immunostained with an anti-CD31 antibody (Human CD31/PECAM-1 PE-conjugated Antibody, FAB3567P, R&D Systems, Inc.). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined.

In the case of the formation of vascular endothelial networks with either human iPS-derived vascular endothelial cells or human cardiac microvascular endothelial cells, co-culture with human dermal fibroblasts resulted in promoted formation while co-culture with human cardiac fibroblasts resulted in inhibited formation.

### Example 3

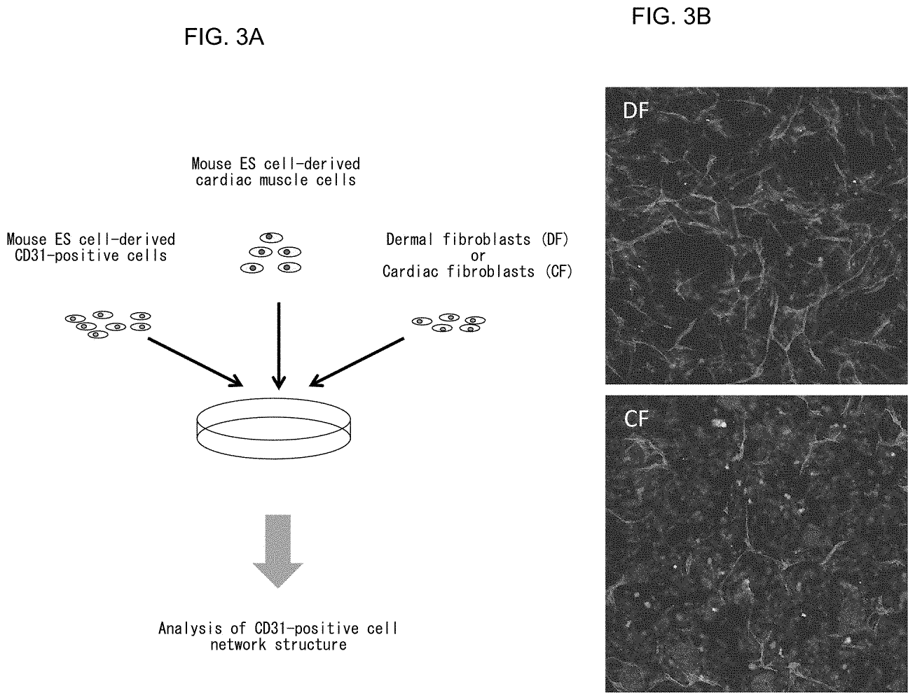

Cardiac Fibroblasts Inhibit Formation of Vascular Endothelial Networks (FIG. 3)

Mouse dermal fibroblasts or cardiac fibroblasts (6×10⁴ cells/cm²), and mouse ES cell-derived cardiac muscle cells (2.4×10⁵ cells/cm²), and mouse ES cell-derived vascular endothelial cells (2.0×10⁴ cells/cm²) were co-cultured for 3 days at 5% CO₂, 37° C., and then immunostained with an anti-CD31 antibody (PE Rat Anti-Mouse CD31, 553373, BD Biosciences). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined.

Formation of vascular endothelial networks by mouse ES cell-derived vascular endothelial cells was promoted in the presence of mouse dermal fibroblasts, but inhibited in the presence of mouse cardiac fibroblasts.

### Example 4

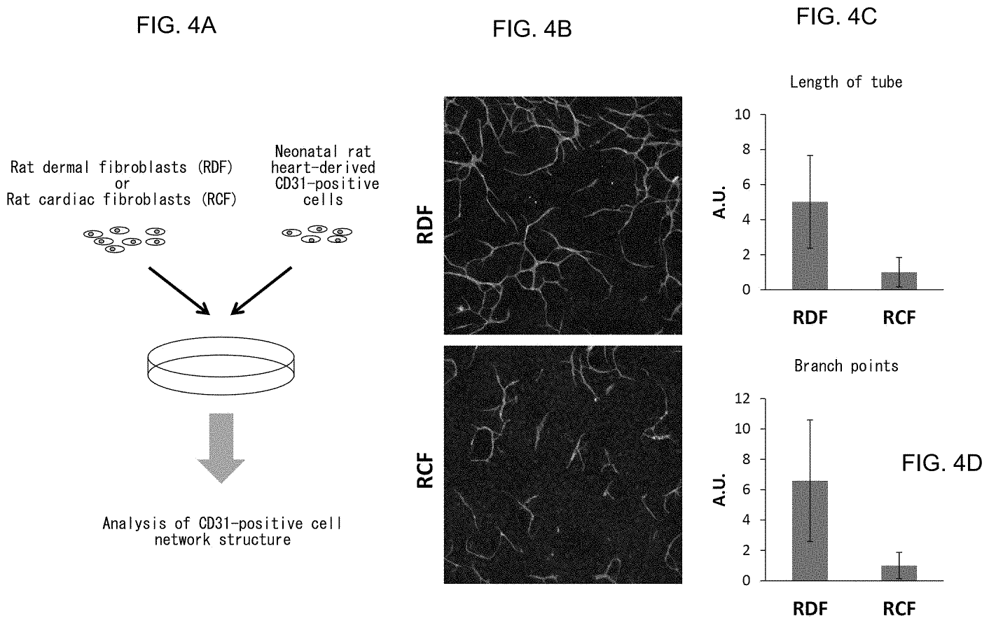

Cardiac Fibroblasts Inhibit Formation of Vascular Endothelial Networks (FIG. 4)

Primary neonatal rat dermal fibroblasts (RDF) or cardiac fibroblasts (RCF) (2.4×10⁵ cells/cm²) obtained from SD rats (Jcl:SD, Sankyo Labo Service Corporation, Japan), and neonatal rat heart-derived vascular endothelial cells (2.0×10⁴ cells/cm²) were co-cultured for 3 days at 5% CO₂, 37° C., and then immunostained with an anti-CD31 antibody (Mouse anti Rat CD31 Antibody, MCA1334G, Bio-Rad). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined.

Formation of vascular endothelial networks was promoted in co-culture with normal rat dermal fibroblasts, but inhibited in co-culture with rat cardiac fibroblasts.

### Example 5

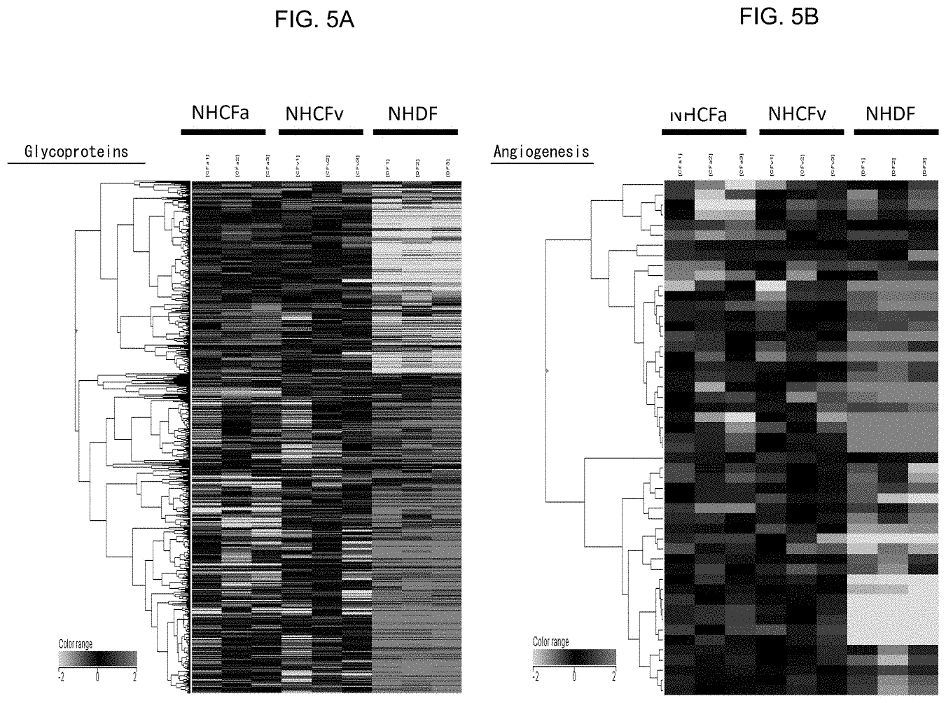

Comparison of Gene Expression Between Dermal Fibroblasts and Cardiac Fibroblasts (FIG. 5)

Total RNA was extracted from human dermal fibroblasts and cardiac fibroblasts (cardiac atrium-derived and cardiac ventricle-derived) and subjected to microarray analysis for gene expression (commissioned to DNA Chip Research Inc. (Japan)). Heat maps for glycoprotein-related genes and angiogenesis-related gene are shown (FIG. 5).

The gene expression patterns of human dermal fibroblasts and cardiac fibroblasts were greatly different. Candidate molecules were screened based on the results of the array to identify LYPD1, an angiogenesis inhibitor that was highly expressed in cardiac fibroblasts (Gen Bank accession no.: NM_144586.6, SEQ ID NO: 1).

### Example 6

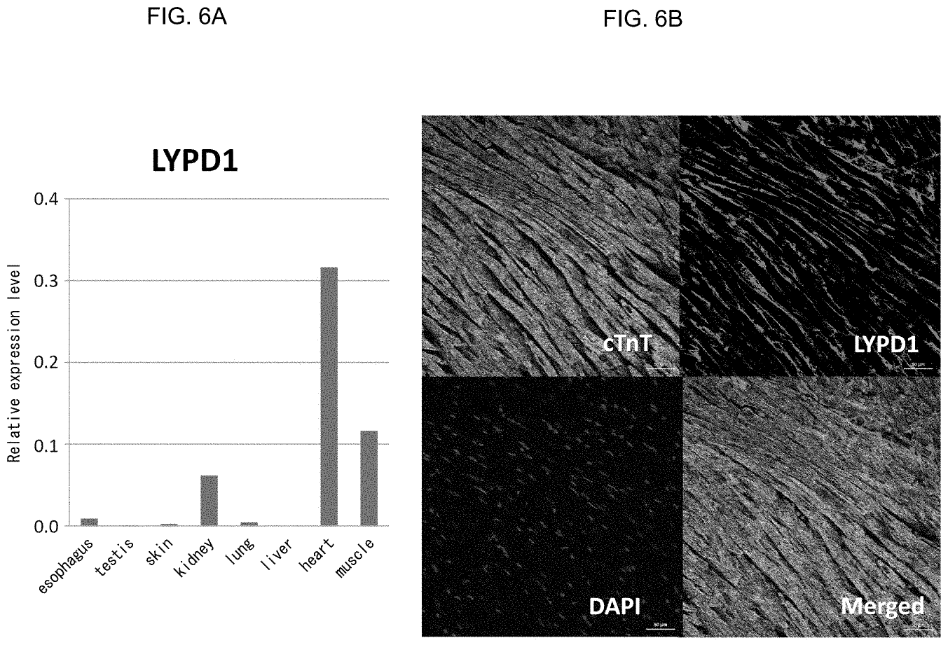

LYPD1 Expresses in Rat Cardiac Stroma (FIG. 6)

The LYPD1 expression in organs from a rat was evaluated by qPCR. Total RNA was extracted from rat organs. mRNA contained in the total RNA fraction was used as a template to synthesize cDNA, which was then used as a template for qPCR. qPCR was performed by a comparative CT method using TaqMan® Gene Expression Assays (Rn01295701_m1, Thermo Fisher Scientific Inc.) (FIG. 6A). Evaluation of the LYPD1 expression in organs from a rat demonstrated that LYPD1 was highly expressed in heart.

FIG. 6B shows immunostained images of a rat cardiac tissue. An anti-cTnT antibody (cardiac Troponin T antibody (Anti-Troponin T, Cardiac Isoform, Mouse-Mono (13-11), AB-1, MS-295-P, Thermo Fisher Scientific Inc.)), an anti-LYPD1 antibody (ab157516, Abcam), and DAPI (nucleus) were used for the staining.

Evaluation of the expression in rat cardiac tissues by immunostaining demonstrated that co-staining in cardiac muscle cells positive for cardiac Troponin T was not observed, but LYPD1 was expressed in cardiac stroma.

### Example 7

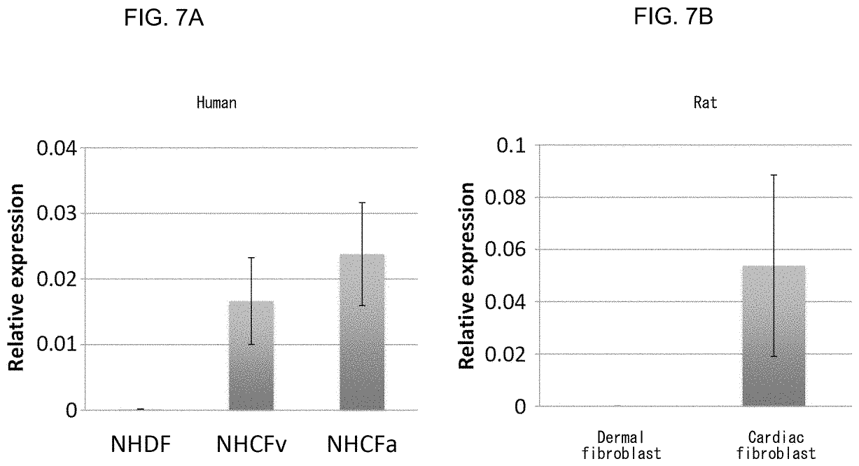

Comparison of the LYPD1 Gene Expression in Human and Rat Primary Cultured Cells (FIG. 7)

The expression of LYPD1 in dermal fibroblasts and cardiac fibroblasts from human and neonatal rats was evaluated by qPCR. Total RNA was extracted from cells. mRNA contained in the total RNA fraction was used as a template to synthesize cDNA, which was then used as a template for qPCR. qPCR was performed by a comparative CT method using TaqMan® Gene Expression Assays (Hs00375991_m1 for human, Rn01295701_m1 for rat, Thermo Fisher Scientific Inc.).

LYPD1 was little detected in denial fibroblasts from human and neonatal rats but highly expressed in cardiac fibroblasts.

### Example 8

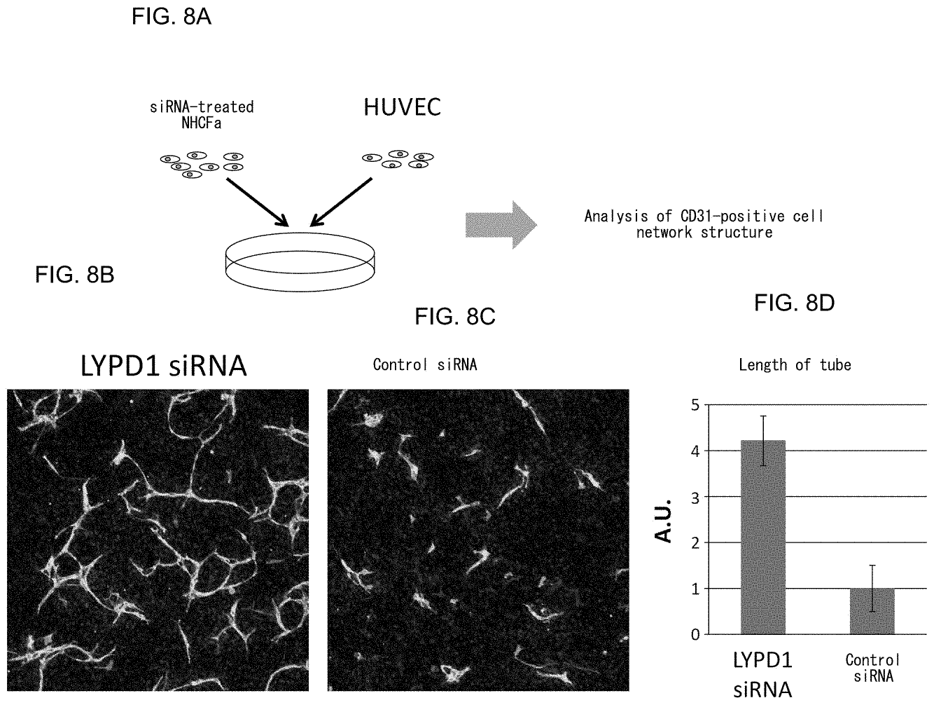

Inhibition of LYPD1 Rescue Vascular Network Formation (FIG. 8)

Using Lipofectamine™ RNAiMAX Transfection Reagent (Thermo Fisher Scientific Inc.), siRNA against LYPD1 (Silencer® Select siRNA, Cat. #4392420, Thermo Fisher Scientific Inc.) (1 nM) or control siRNA (Silencer® Select Negative Control No. 2 siRNA, Cat. #4390846) (1 nM) was transfected into human cardiac fibroblasts. After being cultured for two days, the siRNA-introduced human cardiac fibroblasts (2.4×10⁵ cells/cm²) were co-cultured with HUVECs (2.0×10⁴ cells/cm²) for 3 days at 5% CO₂, 37° C., and then immunostained with an anti-CD31 antibody (Human CD31/PECAM-1 PE-conjugated Antibody, FAB3567P, R&D Systems, Inc.). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of the vascular endothelial networks was determined.

The following sequences can be used as that of siRNA against LYPD1:

(SEQ ID NO: 15)
5′-GGCUUUGCGCUGCAAAUCC-3′;
and
(SEQ ID NO: 16)
5′-GGAUUUGCAGCGCAAAGCC-3′.

The present Example employed the following sequences in order to further increase the stability of the siRNA.

TABLE 1

| SEQ | | |
|---|---|---|
| ID | | |
| NO | Sequence* | note |
| 17 | 5′-GGCUUUGCGCUGCAAAUCCtt-3′ | sense sequence |
| 18 | 5′-GGAUUUGCAGCGCAAAGCCtg-3′ | antisense sequence |

*The 3′ end lower-case characters (tt and tg) represent additional sequences for increasing the stability

The reduced expression of LYPD1 in human cardiac fibroblasts due to siRNA resulted in blocking of the angiogenesis inhibitory effect by LYPD1, and vascular network formation by co-cultured HUVECs (see FIGS. 8B to 8D).

### Example 9

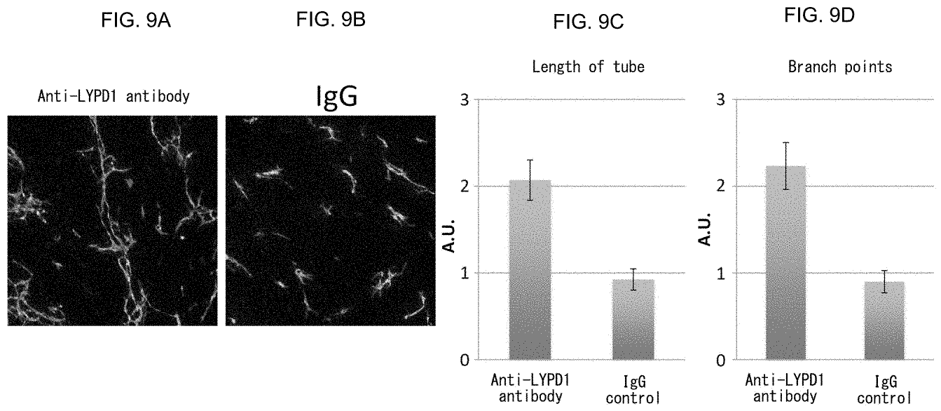

Inhibition of LYPD1 Rescue Vascular Network Formation (FIG. 9)

Human cardiac fibroblasts (2.4×10⁵ cells/cm²) and HUVECs (2.0×10⁴ cells/cm²) were co-cultured in the presence of an anti-LYPD1 antibody (5 μg/mL) (ab157516, Abcam plc.) or a control antibody (5 μg/mL) (normal rabbit IgG, FUJIFILM Wako Pure Chemical Corporation, Japan, Cat. #148-09551) for 4 days at 5% CO₂, 37° C., and then immunostained with an anti-CD31 antibody (Human CD31/PECAM-1 PE-conjugated Antibody, FAB3567P, R&D Systems, Inc.) (FIGS. 9A and 9B). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined (FIGS. 9C and 9D).

The presence of an antibody against LYPD1 resulted in blocking of the angiogenesis inhibitory effect by LYPD1 expressed in human cardiac fibroblasts, and vascular network formation by co-cultured HUVECs.

### Example 10

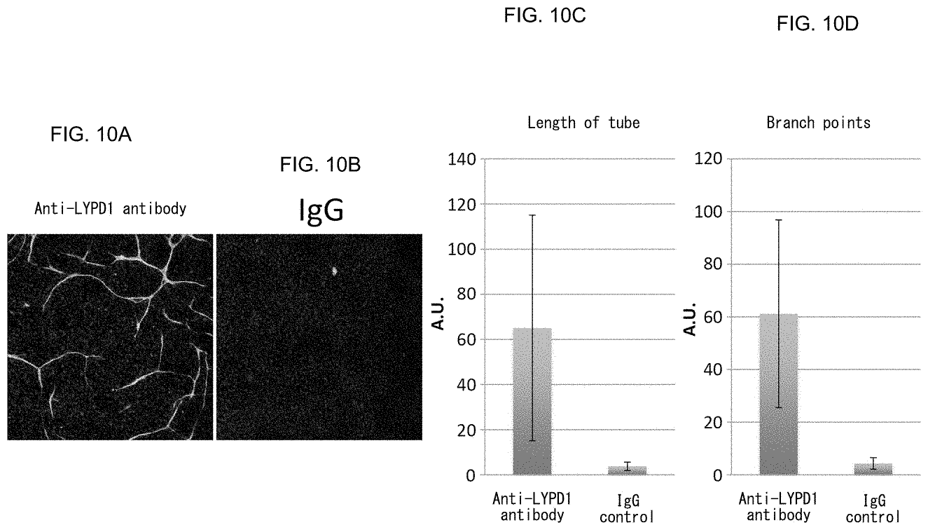

Inhibition of LYPD1 Rescue Vascular Network Formation (FIG. 10)

Neonatal rat cardiac fibroblasts (2.4×10⁵ cells/cm²) and neonatal rat heart-derived vascular endothelial cells (2.0×10⁴ cells/cm²) were co-cultured in the presence of an anti-LYPD1 antibody (5 μg/mL) (ab157516, Abcam plc.) or a control antibody (5 μg/mL) (normal rabbit IgG, FUJIFILM Wako Pure Chemical Corporation, Japan, Cat. #148-09551) for 4 days at 5% CO₂, 37° C., and then immunostained with an anti-CD31 antibody (Mouse anti Rat CD31 Antibody, MCA1334G, Bio-Rad Laboratories, Inc.) (FIGS. 10A and 10B). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of and branch points in the vascular endothelial networks were determined (FIGS. 10C and 10D).

The presence of an antibody against LYPD1 resulted in blocking of the angiogenesis inhibitory effect by LYPD1 expressed in rat cardiac fibroblasts, and vascular network formation by co-cultured rat heart-derived vascular endothelial cells.

### Example 11

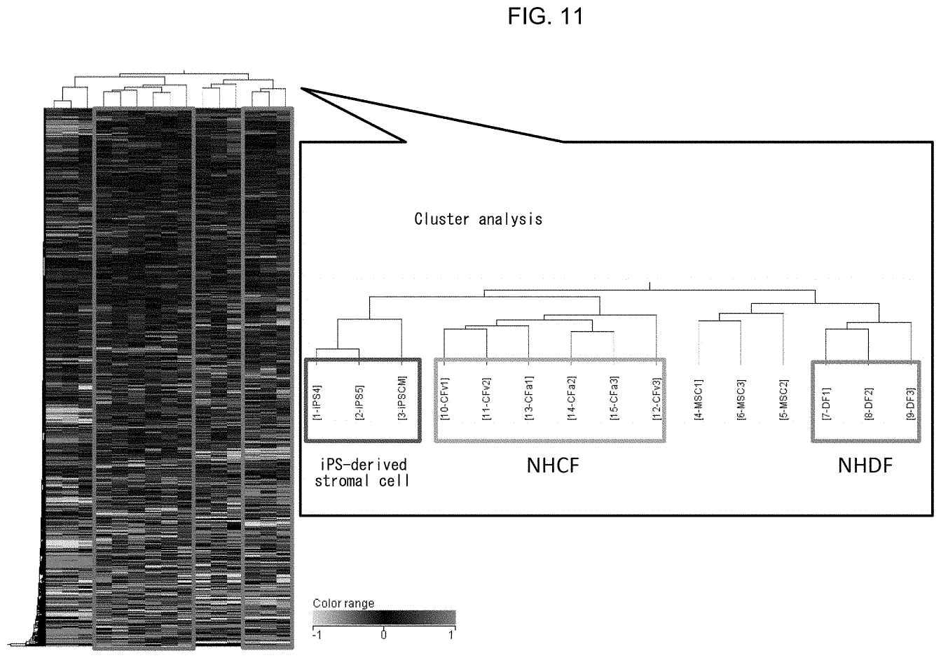

iPS-Derived Stromal Cells are Classified into the Same Cluster as Cardiac Fibroblasts (FIG. 11)

Human dermal fibroblasts (NHDF), human cardiac fibroblasts (NHCF), human iPS-derived stromal cells, and human mesenchymal stem cells (Lonza, Cat. #PT-2501) were analyzed for their gene expression by microarray and genes were clustered. iPS-derived stromal cells were Classified into the same cluster as cardiac fibroblasts.

### Example 12

iPS-Derived Stromal Cells Inhibit Formation of Vascular Endothelial Networks by iPS CD31-Positive Cells (FIG. 12)

Human iPS-derived stromal cells were co-cultured with human iPS CD31-positive cells, and then immunostained with an anti-CD31 antibody (Human CD31/PECAM-1 PE-conjugated Antibody, FAB3567P, R&D Systems, Inc.). ImageXpress Ultra confocal high content screening system (Molecular Devices, LLC, Sunnyvale, Calif., USA) was used to obtain CD31 staining images (FIG. 12B).

Formation of vascular endothelial networks by human iPS CD31-positive cells was promoted in co-culture with human dermal fibroblasts, but inhibited in co-culture with human iPS-derived stromal cells.

The LYPD1 expression in human dermal fibroblasts (NHDF), human cardiac fibroblasts (NHCFa), and human iPS-derived stromal cells (iPS fibro-like) was evaluated by qPCR. Total RNA was extracted from cells. mRNA contained in the total RNA fraction was used as a template to synthesize cDNA, which was then used as a template for qPCR. qPCR was performed by a comparative CT method using TaqMan® Gene Expression Assays (Hs00375991_m1, Thermo Fisher Scientific Inc.) (FIG. 12C).

Human iPS-derived stromal cells highly expressed LYPD1 like human cardiac fibroblasts.

### Example 13

Expression and Purification of Recombinant LYPD1, and Confirmation of the Inhibitory Effect on Vascular Endothelial Networks

The protein coding human LYPD1 cDNA sequence was selected according to published sequence data. Human LYPD1 with FLAG sequence inserted after the signal sequence was synthesized by GenScript Biotech Corporation (Piscataway, N.J., USA), and then inserted into a pcDNA3.1 vector (hereinafter referred to as “pFLAG-LYPD1”).

COS-7 cells were cultured and maintained in DMEM (Dulbecco's modified Eagle medium; Invitrogen) supplemented with 10% fetal bovine serum in an atmosphere at 37° C. and 5% CO₂. pFLAG-LYPD1 was transfected into the COS-7 cells using Lipofectamine® 3000 (Invitrogen) according to the manufacturers' instructions. Forty-eight hours after the transfection, the cells were lysed with RIPA buffer (FUJIFILM Wako Pure Chemical Corporation, Japan).

FLAG-LYPD1 protein was immunoprecipitated using anti-DYKDDDDK tag antibody magnetic beads (FUJIFILM Wako Pure Chemical Corporation, Japan) at 4° C. for 3 hours. After washing the beads with RIPA buffer three times, DYKDDDDK peptide (FUJIFILM Wako Pure Chemical Corporation, Japan) was added to elute FLAG-LYPD1 protein from the beads. The eluate was separated on a 12.5% SDS-PAGE gel and blotted onto Immobilon-P (Merck & Co., Inc., Germany)

FLAG-LYPD1 protein was detected using peroxidase-conjugated anti-DYKDDDDK tag monoclonal antibody (FUJIFILM Wako Pure Chemical Corporation, Japan) and rabbit anti-LYPD1 polyclonal antibody (Abcam plc.).

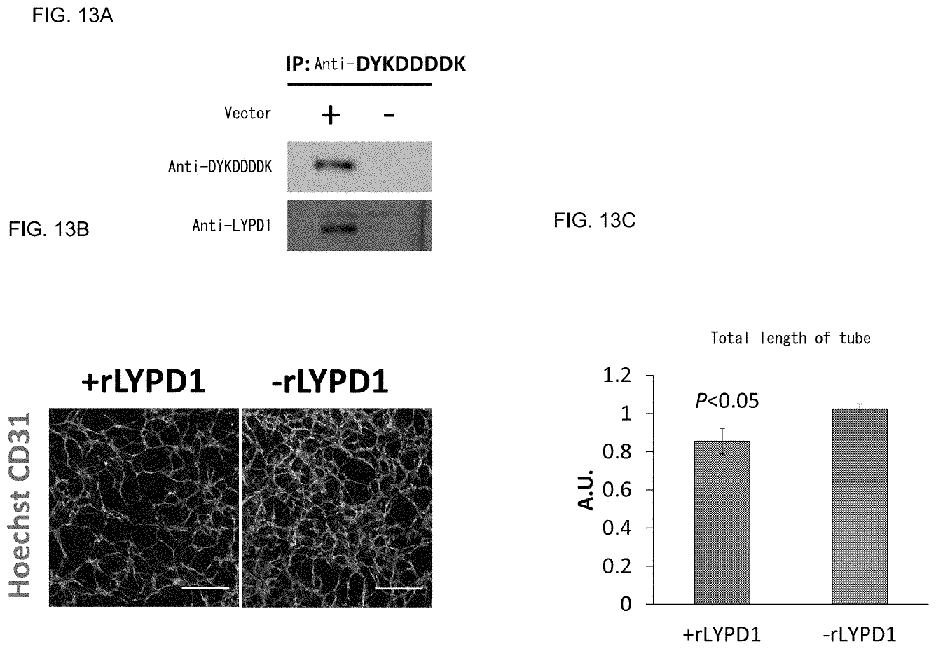

Bands were visualized using ECL Prime Western Blotting Detection Reagent (GE Healthcare UK Ltd., UK) according to the manufacturers' instructions and detected using a digital imaging system (LAS3000, GE Healthcare UK Ltd). The protein yield was determined with Coomassie (Bradford) Protein Assay Kit (Thermo Scientific, Rockford, Ill., US) using bovine serum albumin as a standard (FIG. 13A).

A mixed cell population of human dermal fibroblasts (2.4×10⁵ cells/cm²) and HUVECs (2.0×10⁴ cells/cm²) was treated with FLAG-LYPD1 protein (1.25 μg/mL) or IgG control (1.25 μg/mL, normal rabbit IgG, FUJIFILM Wako Pure Chemical Corporation, Japan, Cat. #148-09551), and cultured in Dulbecco's modified Eagle medium (5% CO₂, 37° C.) supplemented with 10% fetal bovine serum and 1% penicillin/streptomycin. Then, the cells were immunostained with an anti-CD31 antibody (Human CD31/PECAM-1 PE-conjugated Antibody, FAB3567P, R&D Systems, Inc.). ImageXpress Ultra confocal high content screening system (Molecular Devices, LLC, Sunnyvale, Calif., USA) was used to obtain CD31 staining images. Using a MetaXpress software (Molecular Devices, LLC) and considering the region stained with an anti-CD31 antibody as vascular endothelial cells, the length of the vascular endothelial networks was determined.

The results demonstrated that addition of recombinant LYPD1 protein resulted in inhibition of the formation of vascular endothelial networks (FIGS. 13B and 13C).

### Example 14

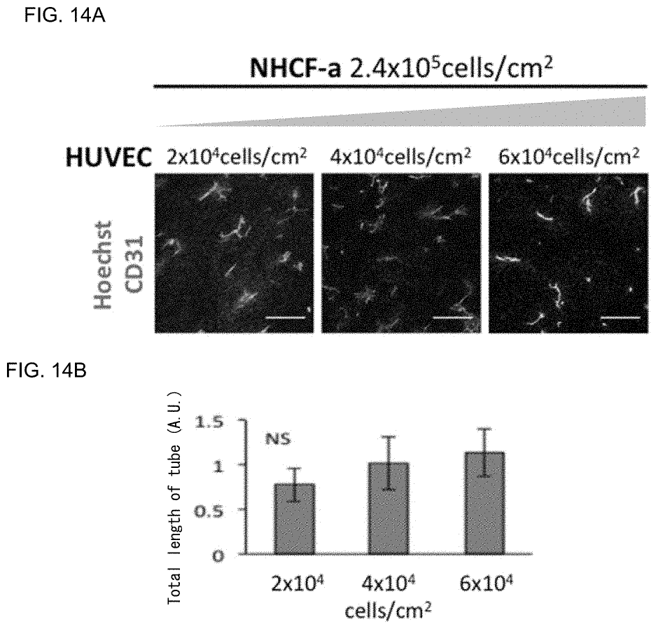

Inhibitory Effect on Angiogenesis by Cardiac Fibroblasts Does Not Depend on Number of Vascular Endothelial Cells (FIG. 14)

Human cardiac atrium-derived fibroblasts (NHCF-a) (2×10⁴ cells/cm², 4×10⁴ cells/cm², and 6×10⁴ cells/cm²) and human umbilical vein endothelial cells (HUVEC) (2.4×10⁵ cells/cm²) were co-cultured for 3 days at 5% CO₂, 37° C., and then immunostained with an anti-CD31 antibody (Human CD31/PECAM-1 PE-conjugated Antibody, FAB3567P, R&D Systems, Inc.), with their nuclei stained with Hoechst. ImageXpress Ultra confocal high content screening system (Molecular Devices, LLC, Sunnyvale, Calif., USA) was used to obtain CD31 staining images and Hoechst fluorescence images. Using a MetaXpress software (Molecular Devices, LLC) and considering the region stained with an anti-CD31 antibody as vascular endothelial cells, the length of and branch points in the vascular endothelial networks were determined (FIGS. 14A and 14B).

The results demonstrated that the inhibitory effect of cardiac fibroblasts on angiogenesis did not depend on the number of vascular endothelial cells.

### Example 15

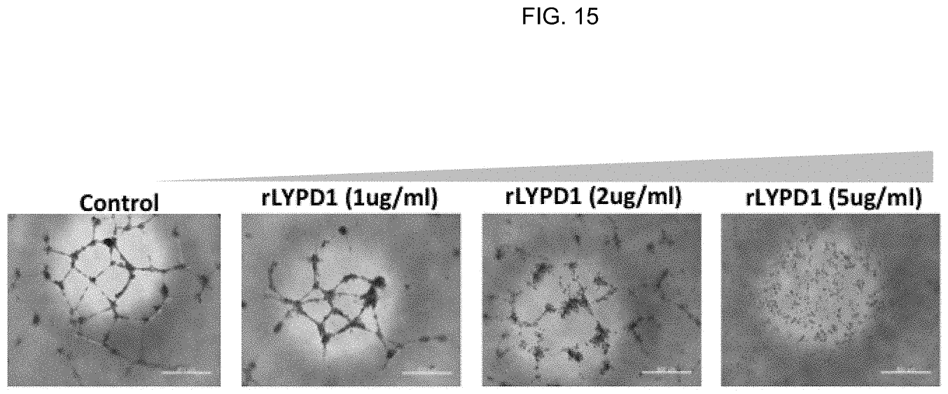

Confirmation of Inhibitory Effect of Recombinant LYPD1 on Formation of Vascular Endothelial Networks in Matrigel® Tube Formation Assay (FIG. 15)

Recombinant LYPD1 protein obtained by the same method as in Example 13 was used in this experiment.

HUVECs (1.0×10⁴ cells/cm²) have been plated on wells in a 96-well plate pre-coated with Matrigel® (BD Biosciences) with reduced growth factors, and cultured using EGM-2 medium (Lonza) in the absence (control) or presence (1 μg/mL, 2 μg/mL, or 5 μg/mL) of recombinant LYPD1 protein for 20 hours (5% CO₂, 37° C.). Thereafter, the tube formation was observed by light microscopy (FIG. 15).

The results demonstrated that recombinant LYPD1 protein directly influenced on HUVECs and successfully inhibited the formation of vascular endothelial networks in a dose-dependent manner.

1. An angiogenesis inhibitor comprising, as an active ingredient, LYPD1 protein or a derivative thereof, or a part thereof; or a vector for expressing the same; or a cell expressing the same.

2. The angiogenesis inhibitor according to claim 1 for use in treatment or prevention of an angiogenesis-related disease.

3. The angiogenesis inhibitor according to claim 2, wherein the angiogenesis-related disease is solid cancer, diabetic retinopathy, age-related macular degeneration, retinopathy of prematurity, corneal graft rejection, neovascular glaucoma, erythroderma, proliferative retinopathy, psoriasis, hemophilic arthropathy, capillary proliferation in atherosclerotic plaques, keloid, wound granulation, vascular adhesion, rheumatoid arthritis, osteoarthritis, an autoimmune disease, a Crohn's disease, restenosis, atherosclerosis, intestinal adhesion, ulcer, liver cirrhosis, glomerulonephritis, diabetic nephropathy, malignant nephrosclerosis, thrombotic microangiopathy, organ graft rejection, glomerulopathy, diabetes mellitus, inflammation, or a neurodegenerative disease.

4. The angiogenesis inhibitor according to claim 3, wherein the solid cancer is cervical cancer, lung cancer, pancreatic cancer, non-small-cell lung cancer, liver cancer, colon cancer, osteosarcoma, skin cancer, head cancer, neck cancer, cutaneous melanoma, intraocular melanoma, uterine cancer, ovarian cancer, rectal cancer, liver cancer, brain tumor, bladder cancer, gastric cancer, perianal gland cancer, colon cancer, breast cancer, fallopian tube cancer, endometrial cancer, vaginal cancer, vulvar cancer, Hodgkin's lymphoma, esophageal cancer, small intestine cancer, endocrine cancer, thyroid cancer, parathyroid cancer, adrenal cancer, soft tissue sarcoma, urethral cancer, penile cancer, prostate cancer, bladder cancer, kidney cancer, ureter cancer, renal cell cancer, renal pelvic cancer, central nervous system (CNS) tumor, primary CNS lymphoma, spinal cord tumor, brainstem glioma, or pituitary adenoma.

5. The angiogenesis inhibitor according to claim 1, wherein the LYPD1 protein has a sequence selected from SEQ ID NOS: 1 to 14 and 19, or has at least 85% sequence identity with a sequence selected from SEQ ID NOS: 1 to 14 and 19.

6. The angiogenesis inhibitor according to claim 1, wherein the cell expresses a higher amount of LYPD protein than skin-derived fibroblasts.

7. The angiogenesis inhibitor according to claim 6, wherein the cell is a heart-derived fibroblast.

8. A method for screening angiogenesis inhibitors that enhance the expression of LYPD1 protein, comprising:
a step (i) of treating and culturing a first cell with a test substance; and
a step (ii) of detecting the expression level of LYPD1 protein in the first cell and comparing it with that of an untreated first cell.

9. The method according to claim 8, wherein the first cell is a fibroblast derived from skin, esophagus, testis, lung, or liver.

10. The method according to claim 8, further comprising:
a step (iii) of selecting the test substance that enhances the expression of LYPD1 protein as compared with the level of LYPD1 protein in the untreated first cell in the step (ii);
a step (iv) of adding the test substance to a cell population comprising a second cell and a vascular endothelial cell and/or precursor cell, and culturing the cell population; and
a step (v) of detecting vascular endothelial networks formed by the vascular endothelial cell and/or precursor cell.

11. The method according to claim 10, wherein the second cell is a fibroblast derived from skin, esophagus, testis, lung, or liver.


SEQUENCE LISTING (19 sequences)

SEQ ID NO: 1 - DNA, 3557 bp, Homo sapiens
  CDS (506)..(775): Homo sapiens LY6/PLAUR domain containing 1 (LYPD1), transcript variant 2, mRNA
gaaatgaaagaagggggagagggagggagggagggagagaaggagcctgaggcagatatggaacgatctctgcagaggaattacaaagaaagtcatttaagcccctttacagaccagctctgctgggaggatgcagaaataaatgatcagcgtcccggcgtgagagagctgcccccagaagtggccgggggagaaaagggtgaagaggagctaaagcttaggaggcgatttccacggaaatggatgctccctaaggatgtgcggaaaggggatgggaatgtgcggattgggggcgggggcaccaacgcactccggcgggcaagcggtctcccgcaccacctgctgcaaggctcgggaaggaaggcgaagatagcaccggggcatccccgggagtccgcaaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtatttataccagttgatgtctgttaattttaaaaaaatgttttcattggtgtttgtttgcgtatccagaaagcagttcatgttatccataaatctggttttgtctttttttgttttaaagaaaaagatgtatacatacagtatagctgcattagataaagcagtgtttgtattttaaaggatgtctgcacaaagaagacctagtgatatttttaaatcaaatggaagaagtgtccctttggcaacaaagcagcatatttaatgacactggttttgcattcagtttcaggggaagcaaagtcaggaatagcctgtcgccaagaatgttttttggacatatacatactaggtatgcacacctataatcatgatgctcatatctgcaacagcatatgtgtttcttttcagacacttttagatccctcatgtggggaaaaagaattattcagagatggcaaatataaaacttccttctagttcagccagtaacatgttcccttcctttgcagcactgagctgtgctgtcaacagcccagaagcaatcaggccctagagaagagaccactcaaaggcccttctgtagatcaaatgtttactgcatgtacatttgtttgcatgcccacatatttgtattccaacttaagtaaccaccaccagttctgcaattctgactgacagagataaagatgctacatagaccacaaacaactgaaatcacaggtatcatgagagtttagttacagtgacaaaagcaaaaaagaacaaaggaagatcaggggatctgtgaagcatttgctctctcttttcgtaaggagccaagacacccacagtaaattcccctgtagagagctgctaccttaaagcaggatttgcattttcagaaatgcttccttcctctcctacatttcaatcgtagtaagaaacatttactcacattttcaatcttctgattttctagaaaccctagggaagtgacagttggcaatgaatgcttcctgcctatgacccatggtaaatattctattaataaatgggggccagacatggtggcgcatgcctgatatctcaatactctgggaggccaaggcagaaggatcacttaagcctagaaatttgagacccacctaggcaacatagcaagaccccatctctacaaaaaaagaaaaacttagccaggcatggtggtacatacacacctgtggtctcagatactttttgggggctgaggcgggaggatcacttgagcccaggaggtgaaggctacagtgagacacgaatgtgccactgcactccagcctggctgacagagtgaaactgtctcaataaaccaataaataaatgctccaggaaaaaacagccacattcacacatccagaattgagcctcctgtatgcactggcctgagtattccttgcctgctgttggaggggaccctagctgtgttcaaatcctccacaaatccatatgtgagcaaggaaggccttggaaactcttctcctttgttaatttccacaggtttctcctgtcaactcccagcctaaaactttgaaatataagccaatttgtttattttttaaaaaaaaaaaaaaaaa

SEQ ID NO: 2 - protein, 89 aa, Homo sapiens
MCQKEVMEQSAGIMYRKSCASSAACLIASAGYQSFCSPGKLNSVCISCCNTPLCNGPRPKKRGSSASALRPGLRTTILFLKLALFSAHC

SEQ ID NO: 3 - DNA, 3646 bp, Homo sapiens
  CDS (439)..(864): Homo sapiens LY6/PLAUR domain containing 1 (LYPD1), transcript variant 1, mRNA
gcgggagaagaggaagacaggaagggggtggggatgtgaagcgaccgtcccagccttccccgcccgccacccccaccccaactcggcagccgtcacgtgatgcctggagtgggaggtggggagaaaaggcgagacttttgtgggtgctcccgatcgccagtagttccttcagtctcagccgccaactccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtatttataccagttgatgtctgttaattttaaaaaaatgttttcattggtgtttgtttgcgtatccagaaagcagttcatgttatccataaatctggttttgtctttttttgttttaaagaaaaagatgtatacatacagtatagctgcattagataaagcagtgtttgtattttaaaggatgtctgcacaaagaagacctagtgatatttttaaatcaaatggaagaagtgtccctttggcaacaaagcagcatatttaatgacactggttttgcattcagtttcaggggaagcaaagtcaggaatagcctgtcgccaagaatgttttttggacatatacatactaggtatgcacacctataatcatgatgctcatatctgcaacagcatatgtgtttcttttcagacacttttagatccctcatgtggggaaaaagaattattcagagatggcaaatataaaacttccttctagttcagccagtaacatgttcccttcctttgcagcactgagctgtgctgtcaacagcccagaagcaatcaggccctagagaagagaccactcaaaggcccttctgtagatcaaatgtttactgcatgtacatttgtttgcatgcccacatatttgtattccaacttaagtaaccaccaccagttctgcaattctgactgacagagataaagatgctacatagaccacaaacaactgaaatcacaggtatcatgagagtttagttacagtgacaaaagcaaaaaagaacaaaggaagatcaggggatctgtgaagcatttgctctctcttttcgtaaggagccaagacacccacagtaaattcccctgtagagagctgctaccttaaagcaggatttgcattttcagaaatgcttccttcctctcctacatttcaatcgtagtaagaaacatttactcacattttcaatcttctgattttctagaaaccctagggaagtgacagttggcaatgaatgcttcctgcctatgacccatggtaaatattctattaataaatgggggccagacatggtggcgcatgcctgatatctcaatactctgggaggccaaggcagaaggatcacttaagcctagaaatttgagacccacctaggcaacatagcaagaccccatctctacaaaaaaagaaaaacttagccaggcatggtggtacatacacacctgtggtctcagatactttttgggggctgaggcgggaggatcacttgagcccaggaggtgaaggctacagtgagacacgaatgtgccactgcactccagcctggctgacagagtgaaactgtctcaataaaccaataaataaatgctccaggaaaaaacagccacattcacacatccagaattgagcctcctgtatgcactggcctgagtattccttgcctgctgttggaggggaccctagctgtgttcaaatcctccacaaatccatatgtgagcaaggaaggccttggaaactcttctcctttgttaatttccacaggtttctcctgtcaactcccagcctaaaactttgaaatataagccaatttgtttattttttaaaaaaaaaaaaaaaaa

SEQ ID NO: 4 - protein, 141 aa, Homo sapiens
MWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRPGLRTTILFLKLALFSAHC

SEQ ID NO: 5 - DNA, 3519 bp, Homo sapiens
  CDS (264)..(737): Homo sapiens LY6/PLAUR domain containing 1 (LYPD1), transcript variant 3, mRNA
gcgggagaagaggaagacaggaagggggtggggatgtgaagcgaccgtcccagccttccccgcccgccacccccaccccaactcggcagccgtcacgtgatgcctggagtgggaggtggggagaaaaggcgagacttttgtgggtgctcccgatcgccagtagttccttcagtctcagccgccaactccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtatttataccagttgatgtctgttaattttaaaaaaatgttttcattggtgtttgtttgcgtatccagaaagcagttcatgttatccataaatctggttttgtctttttttgttttaaagaaaaagatgtatacatacagtatagctgcattagataaagcagtgtttgtattttaaaggatgtctgcacaaagaagacctagtgatatttttaaatcaaatggaagaagtgtccctttggcaacaaagcagcatatttaatgacactggttttgcattcagtttcaggggaagcaaagtcaggaatagcctgtcgccaagaatgttttttggacatatacatactaggtatgcacacctataatcatgatgctcatatctgcaacagcatatgtgtttcttttcagacacttttagatccctcatgtggggaaaaagaattattcagagatggcaaatataaaacttccttctagttcagccagtaacatgttcccttcctttgcagcactgagctgtgctgtcaacagcccagaagcaatcaggccctagagaagagaccactcaaaggcccttctgtagatcaaatgtttactgcatgtacatttgtttgcatgcccacatatttgtattccaacttaagtaaccaccaccagttctgcaattctgactgacagagataaagatgctacatagaccacaaacaactgaaatcacaggtatcatgagagtttagttacagtgacaaaagcaaaaaagaacaaaggaagatcaggggatctgtgaagcatttgctctctcttttcgtaaggagccaagacacccacagtaaattcccctgtagagagctgctaccttaaagcaggatttgcattttcagaaatgcttccttcctctcctacatttcaatcgtagtaagaaacatttactcacattttcaatcttctgattttctagaaaccctagggaagtgacagttggcaatgaatgcttcctgcctatgacccatggtaaatattctattaataaatgggggccagacatggtggcgcatgcctgatatctcaatactctgggaggccaaggcagaaggatcacttaagcctagaaatttgagacccacctaggcaacatagcaagaccccatctctacaaaaaaagaaaaacttagccaggcatggtggtacatacacacctgtggtctcagatactttttgggggctgaggcgggaggatcacttgagcccaggaggtgaaggctacagtgagacacgaatgtgccactgcactccagcctggctgacagagtgaaactgtctcaataaaccaataaataaatgctccaggaaaaaacagccacattcacacatccagaattgagcctcctgtatgcactggcctgagtattccttgcctgctgttggaggggaccctagctgtgttcaaatcctccacaaatccatatgtgagcaaggaaggccttggaaactcttctcctttgttaatttccacaggtttctcctgtcaactcccagcctaaaactttgaaatataagccaatttgtttattttttaaaaaaaaaaaaaaaaa

SEQ ID NO: 6 - protein, 157 aa, Homo sapiens
MQAPRAAPAAPLSYDRRLRGSIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRPGLRTTILFLKLALFSAHC

SEQ ID NO: 7 - DNA, 3680 bp, Homo sapiens
  CDS (629)..(898): Homo sapiens LY6/PLAUR domain containing 1 (LYPD1), transcript variant 4, mRNA
gcgggagaagaggaagacaggaagggggtggggatgtgaagcgaccgtcccagccttccccgcccgccacccccaccccaactcggcagccgtcacgtgatgcctggagtgggaggtggggagaaaaggcgagacttttgtgggtgctcccgatcgccagtagttccttcagtctcagccgccaactccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggtgagaatacccagaggccagcagccgaggccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtatttataccagttgatgtctgttaattttaaaaaaatgttttcattggtgtttgtttgcgtatccagaaagcagttcatgttatccataaatctggttttgtctttttttgttttaaagaaaaagatgtatacatacagtatagctgcattagataaagcagtgtttgtattttaaaggatgtctgcacaaagaagacctagtgatatttttaaatcaaatggaagaagtgtccctttggcaacaaagcagcatatttaatgacactggttttgcattcagtttcaggggaagcaaagtcaggaatagcctgtcgccaagaatgttttttggacatatacatactaggtatgcacacctataatcatgatgctcatatctgcaacagcatatgtgtttcttttcagacacttttagatccctcatgtggggaaaaagaattattcagagatggcaaatataaaacttccttctagttcagccagtaacatgttcccttcctttgcagcactgagctgtgctgtcaacagcccagaagcaatcaggccctagagaagagaccactcaaaggcccttctgtagatcaaatgtttactgcatgtacatttgtttgcatgcccacatatttgtattccaacttaagtaaccaccaccagttctgcaattctgactgacagagataaagatgctacatagaccacaaacaactgaaatcacaggtatcatgagagtttagttacagtgacaaaagcaaaaaagaacaaaggaagatcaggggatctgtgaagcatttgctctctcttttcgtaaggagccaagacacccacagtaaattcccctgtagagagctgctaccttaaagcaggatttgcattttcagaaatgcttccttcctctcctacatttcaatcgtagtaagaaacatttactcacattttcaatcttctgattttctagaaaccctagggaagtgacagttggcaatgaatgcttcctgcctatgacccatggtaaatattctattaataaatgggggccagacatggtggcgcatgcctgatatctcaatactctgggaggccaaggcagaaggatcacttaagcctagaaatttgagacccacctaggcaacatagcaagaccccatctctacaaaaaaagaaaaacttagccaggcatggtggtacatacacacctgtggtctcagatactttttgggggctgaggcgggaggatcacttgagcccaggaggtgaaggctacagtgagacacgaatgtgccactgcactccagcctggctgacagagtgaaactgtctcaataaaccaataaataaatgctccaggaaaaaacagccacattcacacatccagaattgagcctcctgtatgcactggcctgagtattccttgcctgctgttggaggggaccctagctgtgttcaaatcctccacaaatccatatgtgagcaaggaaggccttggaaactcttctcctttgttaatttccacaggtttctcctgtcaactcccagcctaaaactttgaaatataagccaatttgtttattttttaaaaaaaaaaaaaaaaa

SEQ ID NO: 8 - protein, 89 aa, Homo sapiens; identical to SEQ ID NO: 2

SEQ ID NO: 9 - DNA, 6480 bp, Mus musculus
  CDS (325)..(750): Mus musculus Ly6/Plaur domain containing 1 (Lypd1), transcript variant 1, mRNA
agaagaggcgagacttttttgggtgctccggatcgccagtagttcttcaagcctcagcagccaactcctccggaggcgctgcgctccgccccagggagcgcgaatccaaggagcctgggaccagcctctgggagcccccggcgcgggcgatgcgggcgccgcgggcgacacctgcggctcctctcggtggcagccgtcgcttgggcggcagcagcgcgagcctcggcagcctcggcagctactgtcgccgcggccagaacagcctccgctgcggtcgtggtctctgatgctcttgcccgctcccggccctgccgatccgggaggatgtgggttctcggcatcgcagcaactttttgcggattgttctggcttccagggctggcgctgcaaattcagtgctaccagtgtgaagaattccagctgaacaacgattgctcatcccctgagttcatcgtaaattgcaccgtgaacgttcaagacatgtgtcagaaagaagtgatggagcaaagtgctgggatcatgtaccggaagtcgtgtgcatcgtcagcagcctgtctcattgcttcagctgggtaccagtccttctgttcccctgggaaactgaactccgtgtgcatcagctgctgcaacacccctctttgcaatgggccgaggcccaagaagagaggcagctctgcctcggccatcaggccagggcttctcaccactctcctgttcttccacttagccctctgcttggcacactgctgaagctaaaggagatgccaacccctgctgcctcacctgtctggcccttcgtctctcaccttcccgagtctcttctgggtgtccttttattctgggtagacaagggagtctttttgttccctcctttcaagtaacgcaagattgccgtgcacaaatacttttgtaagctctgaaccaattcattctgaatttctgtgtgtagttgaagaaaaaagcatggagcagaaagtccagaccctcccatcccaatctggttaaccaccgccaaggctagcctggaagaaccagcccttagaagtcattgagatacgcatctgcctttcccaaagccttgagcttccattctgtcccagtaggagtcacagtctattcagagactgctgctgcgtgaaggtaactttgcttttgcgggaggggagagccagtttcggctcaaggcttctgaacttgccattcatacttcctgctcctgtaaactattttctggggtggacccagctggtttggtctctgagccagtctgtggtgactcaggactcaagggctggggcttagcctctccaggcttggcctcagtctgaaaagtgcttaagaaaaccttgttagttctcctggaggaagagttactgcgccgggaggctaggaagatgagggggctgcgggctgagctggtgctgtccttggtggagatgaagcgggcacgctggcgtttctcttggttggcatgctgcagagtcaggcggcagcagagcacctgccagaacaccttccggaactgctgagaggacacgttgtagaggagagggttgaccacagagctgaggtagaagaaggtatcagagaagggcaggaggatcatgtatgccctgaagtacgttctggtccagtcatgtttgggttttgctgcagccatgatccgtcggatctgattgggcatccaacacacggccaacgtcaccacaatcagtcctggcaggcaagaacaggagagaaaaggagacggggagagaaacagcatgagaacaaaaataaataaataaaaacccataaaatattaagccccttggttctgttgcttactggccgagaaacggtaccaatctttcagctctgtgcttgtcggcttctttttgccactggcaaaggagaatttaatgctgcttcaagctcaggggacttggctatgttaaaaagcgttaaatgctttcgacagtgtatttatacttacggctgcctgttaattttcaaaatgttttcattgttgctcgtgtatccagaaaatatctcacgttggccataaccctggttttgtctttttgtttctagatgagcccataaggtatagccatagtagagggaaacggtagctgtttttataaacgactactgaactatgtgagaagacacagtgatattgtgaagtcaaatggaagttgtttcattttgcctaaacaaatacattttgtgatgctgattttgcatccagtttcaggagaagtaaaatcagggacaggtagtcatggagaagaggtactactaagccctgcccctgtgcgtgaggcactgagtatccagacatccatatgaatgcccattacattctggcttacataaacacggctacttctaaagtatgacaaatgaaatcacagtacagtgtgtgtgtgtgtgtgtgtgtgtgtgtgtgtgtgtgtgtgtgtgtagaaagtagacgatatataaatgtatgtcccaaaaggtatacaacctccatctatatcagcatctctgcttcttttcaaacacttttggaagccccttccaccttaacttaggaaaacaaattgtccccaaacaccaaaatctcaaaaactcatgccatgtacagacaggggcacaccctccgcttttgtagcactgagctgcagtgtgactggccaggaatgatgaggccccacagaagcagtgctgctcagcccccactgagaatggggcattagctgactggacactcatttgcatacccggagcttacattctggctcccactatcacatcgaatctatagtctgacaagcacctaaaattcacaagactctgactctttattgataaagcaaagaaacaggcctggggaacctttaggatgttacttactcctttttatgagaagctgagcatcatagcaaatgttcccatgagagggctgatgttatttaaagcaagaatttggtagcactgaagagatagctcagcagttaagatgtaagttctgttcccagcacacatcaggtggctcacaatggcctgtcactccagatgctggggtgggggatctctcagcacctgcacttacatgcatatccctccccatacatacataataagcatatatatatatgtatgtgtatatatatatatgtatgtatatatgtatgtatatatgtatgtatatatatttgcattttacaagtacctcttcccactctgagcccaatttgctaggaaactactgggaaataagaacagggtccctgcttcctgcttagggtgtgtggtaaatagctgagtaattaaggacccagggcaagcagacatgtctatgagcccaaggttagcccaagtatcccttgacagtacataactcagctgctcccaaagacagaccctctgaacataacccatctgtcagagggagggcctaggaaactctagtcctctgaacttccaaggattctttctgtcaaacctcagccagggctttgaaatacacaccaatctgcttatttcttccctttgaatttgcttccctgagaacagtcagtaaatctaagaataaaataggcaaagtaagatattgatctgataaaaaaaaaaggctaaataaataaataaaactactatctcaagtcaggagcagattccaaatcaccttgttactactgctggattaaaaagtaacaacaacaacaacaaaagcactcactgttctgattttttaaaaaaatcgcctgcccaggaaactggtactgaggggcttccagctccccaccccgattttttctgaagtttctacagctctgagcgaccagttcagtgttgattgtgaagggaacagatgcctgcgtgcatggggaagctgtgtgttttcacaaagcttcccaggctgcttttgttcctccgtcactgaattctgcagaacaagttcaaccattatccaaggatgtgtttgttagggtatggggatggagcacacagctttctgtgtgtgccatgcgacagacactgtgggacgttgctctttgagaatctagaaagccgactagacgctccctgtacctcccacgggctgaacaagacatagctttgaaagcccaccagcaaaggtagtgagagaaggcaggttgggaaggaggagggtggaggaggaaagattttgggggaggaggggaggaacattagggtggaggagagagatttggggagggatgctgaggagaagtagcatcatggatctcctttcgcattgcactgtctcatctcctgcttatacagacttcccttagcttcctgcatggccttgctcagcctcaatcaccctgaactggtcactccattgaaaggagactttagtttcaaagaaaaaggaatggaataaaagaaagaagaagagaaaaaggaagggaagaaacccagggtctgagtggaagaaagggatggggaagactgaactggagcttgctctgtagtttcagccagggtaaaatcctgactgtaagattgagattgaataacaaatgggagaccatttgaatgagtggcagcaaatagggaacaaaatgagaactccgcaataataagcagatcacaaagctctctaactatagaaagagacaggtcagggggctggagagatggctcagcggttaagagcattgactgctcttccggaggtcctgagttcaaatcccagcaaccacatggtggctcacaaccatctgtaacaagacctgattccctcttctggtatctgaagagacaggttggatgtggaggacagacttggaagagagacagagtctgaatagaaatggacagttgtgactatgccacacaagcaggttctagagacagaaccagggacaagaagtgggaagggaactagcttcttttgctccaggttctgtgctgagccctcacttcccatgtgggtctctcttcttctgacaacccagagaggtcaaaacaatcagtctcttcacggaacaaaggagaaagctgcctatctcccaaacatgtgatacaggctccgttttcctctcaaggtacaatgctatctattggtcccattttcagattgataaagaaacagacaagatggctacagagatggctcagttttcacagtgcctgcttctcaagagttcagatctctggtacccatatgagcactgggtggatatgatgaacctaacagtaataccagtcttggagtgtggatactaaagatttctggagcaacaaggttagtaagactggacagattggtgggctccgaaattgactgagagcccctatctcagtgaatatggaggaggagttaacaaggatgatactcaacatgaacttttgggcctccacatgtgaactcatatgcccacatacaggtaccgacatgggtatgcagacatacatgcatatatgacacatagagagggacgtgaaaattgaaaaaaaaaagtttgacaggcaagaaaatagtaatttatcacaacccatgcataatgtgactctatccactgaaagaacagaagccgtgtatttaatttaaaaatttcagtcaagttaaaaatgaaaaaaaaaaacactcagtgataacaatttttataatgctacctaagtttgtacacatactgctggatttcttaggaaagcgaaatacagagtcatcataaaaattattggtgagctaatttttcttctcctacccccatactaaatcttcaactgtccctttcccaaacatatgcaccaactaccccaaggaactgcccaagtcgcctgtgtttgcaaaactctcaagcactcatgaacaaggattggatttcaatggtttcgactgttttgctagcctctccctagaaaccctctgtaaaagtgatggttaaaatagtaagtctacgttattctgaaagtacaaaactgtcttttgcctgtctcatcataaattgtgtctgtctgcatcgcacaaatcacaccaactgcctggcagagctgttgacatagccctccccccccccccaccttaaatggaggtgagaaaatggctgccattaggtctgtgtttcaaaagataatcttaggaaatctaggaaaatggaatgctgggcctacaggcgccaccaatggcagtctgaggcatgtgcccacgggcattgtgagaatagaggtagcctttctagaacaggaagccaaccagaccctttgtgcaaaatttatcattttacaatgacaggcaattgcttgttgttatatttgaaaaaaaaattgtccttttctacgatacactgttgaggataaataccatactatgtccagagttaactctctattctggctgtttgaaattgtttcagctccactctcagcaaccctttagctaacacaatgctttaaagtcttattcatggcttaatggagtgcattaaagatttgactaaatttatgtttggctgcaaaaaaaaaaaaaaaaaa

SEQ ID NO: 10 - protein, 141 aa, Mus musculus
MWVLGIAATFCGLFWLPGLALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASAIRPGLLTTLLFFHLALCLAHC

SEQ ID NO: 11 - DNA, 6159 bp, Mus musculus
  CDS (160)..(429): Mus musculus Ly6/Plaur domain containing 1 (Lypd1), transcript variant 2, mRNA
agtcagacccatttgcttttcagtgagagcacagatccgggtgtccactccccagggctggcgctgcaaattcagtgctaccagtgtgaagaattccagctgaacaacgattgctcatcccctgagttcatcgtaaattgcaccgtgaacgttcaagacatgtgtcagaaagaagtgatggagcaaagtgctgggatcatgtaccggaagtcgtgtgcatcgtcagcagcctgtctcattgcttcagctgggtaccagtccttctgttcccctgggaaactgaactccgtgtgcatcagctgctgcaacacccctctttgcaatgggccgaggcccaagaagagaggcagctctgcctcggccatcaggccagggcttctcaccactctcctgttcttccacttagccctctgcttggcacactgctgaagctaaaggagatgccaacccctgctgcctcacctgtctggcccttcgtctctcaccttcccgagtctcttctgggtgtccttttattctgggtagacaagggagtctttttgttccctcctttcaagtaacgcaagattgccgtgcacaaatacttttgtaagctctgaaccaattcattctgaatttctgtgtgtagttgaagaaaaaagcatggagcagaaagtccagaccctcccatcccaatctggttaaccaccgccaaggctagcctggaagaaccagcccttagaagtcattgagatacgcatctgcctttcccaaagccttgagcttccattctgtcccagtaggagtcacagtctattcagagactgctgctgcgtgaaggtaactttgcttttgcgggaggggagagccagtttcggctcaaggcttctgaacttgccattcatacttcctgctcctgtaaactattttctggggtggacccagctggtttggtctctgagccagtctgtggtgactcaggactcaagggctggggcttagcctctccaggcttggcctcagtctgaaaagtgcttaagaaaaccttgttagttctcctggaggaagagttactgcgccgggaggctaggaagatgagggggctgcgggctgagctggtgctgtccttggtggagatgaagcgggcacgctggcgtttctcttggttggcatgctgcagagtcaggcggcagcagagcacctgccagaacaccttccggaactgctgagaggacacgttgtagaggagagggttgaccacagagctgaggtagaagaaggtatcagagaagggcaggaggatcatgtatgccctgaagtacgttctggtccagtcatgtttgggttttgctgcagccatgatccgtcggatctgattgggcatccaacacacggccaacgtcaccacaatcagtcctggcaggcaagaacaggagagaaaaggagacggggagagaaacagcatgagaacaaaaataaataaataaaaacccataaaatattaagccccttggttctgttgcttactggccgagaaacggtaccaatctttcagctctgtgcttgtcggcttctttttgccactggcaaaggagaatttaatgctgcttcaagctcaggggacttggctatgttaaaaagcgttaaatgctttcgacagtgtatttatacttacggctgcctgttaattttcaaaatgttttcattgttgctcgtgtatccagaaaatatctcacgttggccataaccctggttttgtctttttgtttctagatgagcccataaggtatagccatagtagagggaaacggtagctgtttttataaacgactactgaactatgtgagaagacacagtgatattgtgaagtcaaatggaagttgtttcattttgcctaaacaaatacattttgtgatgctgattttgcatccagtttcaggagaagtaaaatcagggacaggtagtcatggagaagaggtactactaagccctgcccctgtgcgtgaggcactgagtatccagacatccatatgaatgcccattacattctggcttacataaacacggctacttctaaagtatgacaaatgaaatcacagtacagtgtgtgtgtgtgtgtgtgtgtgtgtgtgtgtgtgtgtgtgtgtgtagaaagtagacgatatataaatgtatgtcccaaaaggtatacaacctccatctatatcagcatctctgcttcttttcaaacacttttggaagccccttccaccttaacttaggaaaacaaattgtccccaaacaccaaaatctcaaaaactcatgccatgtacagacaggggcacaccctccgcttttgtagcactgagctgcagtgtgactggccaggaatgatgaggccccacagaagcagtgctgctcagcccccactgagaatggggcattagctgactggacactcatttgcatacccggagcttacattctggctcccactatcacatcgaatctatagtctgacaagcacctaaaattcacaagactctgactctttattgataaagcaaagaaacaggcctggggaacctttaggatgttacttactcctttttatgagaagctgagcatcatagcaaatgttcccatgagagggctgatgttatttaaagcaagaatttggtagcactgaagagatagctcagcagttaagatgtaagttctgttcccagcacacatcaggtggctcacaatggcctgtcactccagatgctggggtgggggatctctcagcacctgcacttacatgcatatccctccccatacatacataataagcatatatatatatgtatgtgtatatatatatatgtatgtatatatgtatgtatatatgtatgtatatatatttgcattttacaagtacctcttcccactctgagcccaatttgctaggaaactactgggaaataagaacagggtccctgcttcctgcttagggtgtgtggtaaatagctgagtaattaaggacccagggcaagcagacatgtctatgagcccaaggttagcccaagtatcccttgacagtacataactcagctgctcccaaagacagaccctctgaacataacccatctgtcagagggagggcctaggaaactctagtcctctgaacttccaaggattctttctgtcaaacctcagccagggctttgaaatacacaccaatctgcttatttcttccctttgaatttgcttccctgagaacagtcagtaaatctaagaataaaataggcaaagtaagatattgatctgataaaaaaaaaaggctaaataaataaataaaactactatctcaagtcaggagcagattccaaatcaccttgttactactgctggattaaaaagtaacaacaacaacaacaaaagcactcactgttctgattttttaaaaaaatcgcctgcccaggaaactggtactgaggggcttccagctccccaccccgattttttctgaagtttctacagctctgagcgaccagttcagtgttgattgtgaagggaacagatgcctgcgtgcatggggaagctgtgtgttttcacaaagcttcccaggctgcttttgttcctccgtcactgaattctgcagaacaagttcaaccattatccaaggatgtgtttgttagggtatggggatggagcacacagctttctgtgtgtgccatgcgacagacactgtgggacgttgctctttgagaatctagaaagccgactagacgctccctgtacctcccacgggctgaacaagacatagctttgaaagcccaccagcaaaggtagtgagagaaggcaggttgggaaggaggagggtggaggaggaaagattttgggggaggaggggaggaacattagggtggaggagagagatttggggagggatgctgaggagaagtagcatcatggatctcctttcgcattgcactgtctcatctcctgcttatacagacttcccttagcttcctgcatggccttgctcagcctcaatcaccctgaactggtcactccattgaaaggagactttagtttcaaagaaaaaggaatggaataaaagaaagaagaagagaaaaaggaagggaagaaacccagggtctgagtggaagaaagggatggggaagactgaactggagcttgctctgtagtttcagccagggtaaaatcctgactgtaagattgagattgaataacaaatgggagaccatttgaatgagtggcagcaaatagggaacaaaatgagaactccgcaataataagcagatcacaaagctctctaactatagaaagagacaggtcagggggctggagagatggctcagcggttaagagcattgactgctcttccggaggtcctgagttcaaatcccagcaaccacatggtggctcacaaccatctgtaacaagacctgattccctcttctggtatctgaagagacaggttggatgtggaggacagacttggaagagagacagagtctgaatagaaatggacagttgtgactatgccacacaagcaggttctagagacagaaccagggacaagaagtgggaagggaactagcttcttttgctccaggttctgtgctgagccctcacttcccatgtgggtctctcttcttctgacaacccagagaggtcaaaacaatcagtctcttcacggaacaaaggagaaagctgcctatctcccaaacatgtgatacaggctccgttttcctctcaaggtacaatgctatctattggtcccattttcagattgataaagaaacagacaagatggctacagagatggctcagttttcacagtgcctgcttctcaagagttcagatctctggtacccatatgagcactgggtggatatgatgaacctaacagtaataccagtcttggagtgtggatactaaagatttctggagcaacaaggttagtaagactggacagattggtgggctccgaaattgactgagagcccctatctcagtgaatatggaggaggagttaacaaggatgatactcaacatgaacttttgggcctccacatgtgaactcatatgcccacatacaggtaccgacatgggtatgcagacatacatgcatatatgacacatagagagggacgtgaaaattgaaaaaaaaaagtttgacaggcaagaaaatagtaatttatcacaacccatgcataatgtgactctatccactgaaagaacagaagccgtgtatttaatttaaaaatttcagtcaagttaaaaatgaaaaaaaaaaacactcagtgataacaatttttataatgctacctaagtttgtacacatactgctggatttcttaggaaagcgaaatacagagtcatcataaaaattattggtgagctaatttttcttctcctacccccatactaaatcttcaactgtccctttcccaaacatatgcaccaactaccccaaggaactgcccaagtcgcctgtgtttgcaaaactctcaagcactcatgaacaaggattggatttcaatggtttcgactgttttgctagcctctccctagaaaccctctgtaaaagtgatggttaaaatagtaagtctacgttattctgaaagtacaaaactgtcttttgcctgtctcatcataaattgtgtctgtctgcatcgcacaaatcacaccaactgcctggcagagctgttgacatagccctccccccccccccaccttaaatggaggtgagaaaatggctgccattaggtctgtgtttcaaaagataatcttaggaaatctaggaaaatggaatgctgggcctacaggcgccaccaatggcagtctgaggcatgtgcccacgggcattgtgagaatagaggtagcctttctagaacaggaagccaaccagaccctttgtgcaaaatttatcattttacaatgacaggcaattgcttgttgttatatttgaaaaaaaaattgtccttttctacgatacactgttgaggataaataccatactatgtccagagttaactctctattctggctgtttgaaattgtttcagctccactctcagcaaccctttagctaacacaatgctttaaagtcttattcatggcttaatggagtgcattaaagatttgactaaatttatgtttggctgcaaaaaaaaaaaaaaaaaa

SEQ ID NO: 12 - protein, 89 aa, Mus musculus
MCQKEVMEQSAGIMYRKSCASSAACLIASAGYQSFCSPGKLNSVCISCCNTPLCNGPRPKKRGSSASAIRPGLLTTLLFFHLALCLAHC

SEQ ID NO: 13 - DNA, 6189 bp, Mus musculus
  CDS (190)..(459): Mus musculus Ly6/Plaur domain containing 1 (Lypd1), transcript variant 3, mRNA
ttcctgcgatcggtagggtgttaggaaggggcatgcggcccaaagagaagctcccagatcagaggagagcaaccaggaaaatatgggctggcgctgcaaattcagtgctaccagtgtgaagaattccagctgaacaacgattgctcatcccctgagttcatcgtaaattgcaccgtgaacgttcaagacatgtgtcagaaagaagtgatggagcaaagtgctgggatcatgtaccggaagtcgtgtgcatcgtcagcagcctgtctcattgcttcagctgggtaccagtccttctgttcccctgggaaactgaactccgtgtgcatcagctgctgcaacacccctctttgcaatgggccgaggcccaagaagagaggcagctctgcctcggccatcaggccagggcttctcaccactctcctgttcttccacttagccctctgcttggcacactgctgaagctaaaggagatgccaacccctgctgcctcacctgtctggcccttcgtctctcaccttcccgagtctcttctgggtgtccttttattctgggtagacaagggagtctttttgttccctcctttcaagtaacgcaagattgccgtgcacaaatacttttgtaagctctgaaccaattcattctgaatttctgtgtgtagttgaagaaaaaagcatggagcagaaagtccagaccctcccatcccaatctggttaaccaccgccaaggctagcctggaagaaccagcccttagaagtcattgagatacgcatctgcctttcccaaagccttgagcttccattctgtcccagtaggagtcacagtctattcagagactgctgctgcgtgaaggtaactttgcttttgcgggaggggagagccagtttcggctcaaggcttctgaacttgccattcatacttcctgctcctgtaaactattttctggggtggacccagctggtttggtctctgagccagtctgtggtgactcaggactcaagggctggggcttagcctctccaggcttggcctcagtctgaaaagtgcttaagaaaaccttgttagttctcctggaggaagagttactgcgccgggaggctaggaagatgagggggctgcgggctgagctggtgctgtccttggtggagatgaagcgggcacgctggcgtttctcttggttggcatgctgcagagtcaggcggcagcagagcacctgccagaacaccttccggaactgctgagaggacacgttgtagaggagagggttgaccacagagctgaggtagaagaaggtatcagagaagggcaggaggatcatgtatgccctgaagtacgttctggtccagtcatgtttgggttttgctgcagccatgatccgtcggatctgattgggcatccaacacacggccaacgtcaccacaatcagtcctggcaggcaagaacaggagagaaaaggagacggggagagaaacagcatgagaacaaaaataaataaataaaaacccataaaatattaagccccttggttctgttgcttactggccgagaaacggtaccaatctttcagctctgtgcttgtcggcttctttttgccactggcaaaggagaatttaatgctgcttcaagctcaggggacttggctatgttaaaaagcgttaaatgctttcgacagtgtatttatacttacggctgcctgttaattttcaaaatgttttcattgttgctcgtgtatccagaaaatatctcacgttggccataaccctggttttgtctttttgtttctagatgagcccataaggtatagccatagtagagggaaacggtagctgtttttataaacgactactgaactatgtgagaagacacagtgatattgtgaagtcaaatggaagttgtttcattttgcctaaacaaatacattttgtgatgctgattttgcatccagtttcaggagaagtaaaatcagggacaggtagtcatggagaagaggtactactaagccctgcccctgtgcgtgaggcactgagtatccagacatccatatgaatgcccattacattctggcttacataaacacggctacttctaaagtatgacaaatgaaatcacagtacagtgtgtgtgtgtgtgtgtgtgtgtgtgtgtgtgtgtgtgtgtgtgtagaaagtagacgatatataaatgtatgtcccaaaaggtatacaacctccatctatatcagcatctctgcttcttttcaaacacttttggaagccccttccaccttaacttaggaaaacaaattgtccccaaacaccaaaatctcaaaaactcatgccatgtacagacaggggcacaccctccgcttttgtagcactgagctgcagtgtgactggccaggaatgatgaggccccacagaagcagtgctgctcagcccccactgagaatggggcattagctgactggacactcatttgcatacccggagcttacattctggctcccactatcacatcgaatctatagtctgacaagcacctaaaattcacaagactctgactctttattgataaagcaaagaaacaggcctggggaacctttaggatgttacttactcctttttatgagaagctgagcatcatagcaaatgttcccatgagagggctgatgttatttaaagcaagaatttggtagcactgaagagatagctcagcagttaagatgtaagttctgttcccagcacacatcaggtggctcacaatggcctgtcactccagatgctggggtgggggatctctcagcacctgcacttacatgcatatccctccccatacatacataataagcatatatatatatgtatgtgtatatatatatatgtatgtatatatgtatgtatatatgtatgtatatatatttgcattttacaagtacctcttcccactctgagcccaatttgctaggaaactactgggaaataagaacagggtccctgcttcctgcttagggtgtgtggtaaatagctgagtaattaaggacccagggcaagcagacatgtctatgagcccaaggttagcccaagtatcccttgacagtacataactcagctgctcccaaagacagaccctctgaacataacccatctgtcagagggagggcctaggaaactctagtcctctgaacttccaaggattctttctgtcaaacctcagccagggctttgaaatacacaccaatctgcttatttcttccctttgaatttgcttccctgagaacagtcagtaaatctaagaataaaataggcaaagtaagatattgatctgataaaaaaaaaaggctaaataaataaataaaactactatctcaagtcaggagcagattccaaatcaccttgttactactgctggattaaaaagtaacaacaacaacaacaaaagcactcactgttctgattttttaaaaaaatcgcctgcccaggaaactggtactgaggggcttccagctccccaccccgattttttctgaagtttctacagctctgagcgaccagttcagtgttgattgtgaagggaacagatgcctgcgtgcatggggaagctgtgtgttttcacaaagcttcccaggctgcttttgttcctccgtcactgaattctgcagaacaagttcaaccattatccaaggatgtgtttgttagggtatggggatggagcacacagctttctgtgtgtgccatgcgacagacactgtgggacgttgctctttgagaatctagaaagccgactagacgctccctgtacctcccacgggctgaacaagacatagctttgaaagcccaccagcaaaggtagtgagagaaggcaggttgggaaggaggagggtggaggaggaaagattttgggggaggaggggaggaacattagggtggaggagagagatttggggagggatgctgaggagaagtagcatcatggatctcctttcgcattgcactgtctcatctcctgcttatacagacttcccttagcttcctgcatggccttgctcagcctcaatcaccctgaactggtcactccattgaaaggagactttagtttcaaagaaaaaggaatggaataaaagaaagaagaagagaaaaaggaagggaagaaacccagggtctgagtggaagaaagggatggggaagactgaactggagcttgctctgtagtttcagccagggtaaaatcctgactgtaagattgagattgaataacaaatgggagaccatttgaatgagtggcagcaaatagggaacaaaatgagaactccgcaataataagcagatcacaaagctctctaactatagaaagagacaggtcagggggctggagagatggctcagcggttaagagcattgactgctcttccggaggtcctgagttcaaatcccagcaaccacatggtggctcacaaccatctgtaacaagacctgattccctcttctggtatctgaagagacaggttggatgtggaggacagacttggaagagagacagagtctgaatagaaatggacagttgtgactatgccacacaagcaggttctagagacagaaccagggacaagaagtgggaagggaactagcttcttttgctccaggttctgtgctgagccctcacttcccatgtgggtctctcttcttctgacaacccagagaggtcaaaacaatcagtctcttcacggaacaaaggagaaagctgcctatctcccaaacatgtgatacaggctccgttttcctctcaaggtacaatgctatctattggtcccattttcagattgataaagaaacagacaagatggctacagagatggctcagttttcacagtgcctgcttctcaagagttcagatctctggtacccatatgagcactgggtggatatgatgaacctaacagtaataccagtcttggagtgtggatactaaagatttctggagcaacaaggttagtaagactggacagattggtgggctccgaaattgactgagagcccctatctcagtgaatatggaggaggagttaacaaggatgatactcaacatgaacttttgggcctccacatgtgaactcatatgcccacatacaggtaccgacatgggtatgcagacatacatgcatatatgacacatagagagggacgtgaaaattgaaaaaaaaaagtttgacaggcaagaaaatagtaatttatcacaacccatgcataatgtgactctatccactgaaagaacagaagccgtgtatttaatttaaaaatttcagtcaagttaaaaatgaaaaaaaaaaacactcagtgataacaatttttataatgctacctaagtttgtacacatactgctggatttcttaggaaagcgaaatacagagtcatcataaaaattattggtgagctaatttttcttctcctacccccatactaaatcttcaactgtccctttcccaaacatatgcaccaactaccccaaggaactgcccaagtcgcctgtgtttgcaaaactctcaagcactcatgaacaaggattggatttcaatggtttcgactgttttgctagcctctccctagaaaccctctgtaaaagtgatggttaaaatagtaagtctacgttattctgaaagtacaaaactgtcttttgcctgtctcatcataaattgtgtctgtctgcatcgcacaaatcacaccaactgcctggcagagctgttgacatagccctccccccccccccaccttaaatggaggtgagaaaatggctgccattaggtctgtgtttcaaaagataatcttaggaaatctaggaaaatggaatgctgggcctacaggcgccaccaatggcagtctgaggcatgtgcccacgggcattgtgagaatagaggtagcctttctagaacaggaagccaaccagaccctttgtgcaaaatttatcattttacaatgacaggcaattgcttgttgttatatttgaaaaaaaaattgtccttttctacgatacactgttgaggataaataccatactatgtccagagttaactctctattctggctgtttgaaattgtttcagctccactctcagcaaccctttagctaacacaatgctttaaagtcttattcatggcttaatggagtgcattaaagatttgactaaatttatgtttggctgcaaaaaaaaaaaaaaaaaa

SEQ ID NO: 14 - protein, 89 aa, Mus musculus; identical to SEQ ID NO: 12

SEQ ID NO: 15 - RNA, 19 nt, Artificial Sequence
  Oligonucleotide
ggcuuugcgcugcaaaucc

SEQ ID NO: 16 - RNA, 19 nt, Artificial Sequence
  Oligonucleotide
ggauuugcagcgcaaagcc

SEQ ID NO: 17 - DNA, 21 bp, Artificial Sequence
  Oligonucleotide
ggcuuugcgcugcaaaucctt

SEQ ID NO: 18 - DNA, 21 bp, Artificial Sequence
  Oligonucleotide
ggauuugcagcgcaaagcctg

SEQ ID NO: 19 - protein, 164 aa, Homo sapiens
MQAPRAAPAAPLSYDRRPRDSGRMWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRPGLRTTILFLKLALFSAHC